(19) United States
(12) Patent Application Publication
(10) Pub. No.: US 2026/0000657 A1
(43) Pub. Date: Jan. 1, 2026

(54) ANTI-ANGIOGENIC THERAPY AS TREATMENT FOR BENIGN UTERINE DISORDERS

(71) Applicant: STICHTING AMSTERDAM UMC, Amsterdam (NL)

(72) Inventors: Arjan Willem GRIFFIOEN, Amsterdam (NL); Judith Anne Francisca HUIRNE, Amsterdam (NL)

(21) Appl. No.: 18/879,289

(22) PCT Filed: Jun. 28, 2023
(86) PCT No.: PCT/NL2023/050352

§ 371 Date: Dec. 27, 2024

(30) Foreign Application Priority Data
Jun. 28, 2022 (EP) 22181490.8

(51) Int. Cl. A61K 31/4439; A61K 9/00; A61K 31/404; A61K 31/4545; A61K 31/47; A61K 31/5025; A61K 31/506; A61K 31/517; A61K 31/57; A61K 39/00; A61P 15/00; C07K 16/22

(52) U.S. Cl. CPC A61K 31/4439; A61K 9/0036; A61K 31/404; A61K 31/4545; A61K 31/47; A61K 31/5025; A61K 31/506; A61K 31/517; A61K 31/57; A61P 15/00; C07K 16/22; A61K 2039/505

(57) ABSTRACT

The disclosure provides methods and devices for the treating or preventing a benign uterine disorder in a female mammalian subject. The methods comprise administering a non-steroidal angiogenesis inhibitor. Such methods and devices are particularly useful for treating women suffering from adenomyosis and related symptoms.

### FIELD OF THE INVENTION

The disclosure provides methods and devices for the treating or preventing a benign uterine disorder in a female mammalian subject. The methods comprise administering a non-steroidal angiogenesis inhibitor. Such methods and devices are particularly useful for treating women suffering from adenomyosis and related symptoms.

### BACKGROUND OF THE INVENTION

Adenomyosis is a benign condition where endometrium tissue is present in the myometrium layer of the uterus (Bird, McElin et al. 1972). This leads to clinical symptoms of abnormal uterine bleeding, pain, and reduced fertility, with a considerable impact on women's quality of life and subsequently on society (Taran, Stewart et al. 2013, Critchley, Babayev et al. 2020). The etiology of adenomyosis has not been fully clarified. Whereas in the past this condition was thought to mainly develop in multiparous women, improved diagnostic methods have shown that it is also present in nulliparous women and it is associated with a 28% lower clinical pregnancy rate as well as a more than doubled risk of miscarriage in women undergoing IVF (Martinez-Conejero, Morgan et al. 2011, Tremellen and Russell 2011, Vercellini, Consonni et al. 2014). Treatment of adenomyosis is important not only to reduce symptoms, but also to possibly restore fertility. Up to now, we can only treat symptoms of adenomyosis by suppressing the menstrual cycle with hormonal therapy. However, many women experience too many side effects and no full relief of symptoms, and have to turn to more radical therapy such as embolization (only available in research setting) or hysterectomy (Pontis, D'Alterio et al. 2016), both are incompatible with a child wish or restoring fertility.

It is an object of the present disclosure to provide treatments for adenomyosis and other benign uterine disorders.

### SUMMARY OF THE INVENTION

The disclosure provides the following preferred embodiments. However, the invention is not limited to these embodiments.

In some embodiments the disclosure provides a non-steroidal angiogenesis inhibitor for use in treating or preventing a benign uterine disorder in a female mammalian subject, comprising locally administering to a subject in need thereof said non-steroidal angiogenesis inhibitor. The disclosure thus provides methods for treating or preventing a benign uterine disorder in a female mammalian subject. The disclosure further provides a non-steroidal angiogenesis inhibitor for use as a medicament.

In some embodiments the disclosure provides, the inhibitor for use as described herein, wherein the female mammalian subject is afflicted with benign uterine disorder and the method reduces symptoms, increases fertility, and/or increases clinical pregnancy rate and live birth rate.

In some embodiments the disclosure provides the inhibitor for use as described herein, wherein the benign uterine disorder is adenomyosis.

In some embodiments the disclosure provides the inhibitor for use as described herein, wherein the non-steroidal angiogenesis inhibitor is administered intra-uterine.

In some embodiments the disclosure provides the inhibitor for use as described herein, wherein the non-steroidal angiogenesis inhibitor is provided by an intra-uterine device (IUD).

In some embodiments the disclosure provides the inhibitor for use as described herein, wherein the non-steroidal angiogenesis inhibitor is provided as an extended-release formulation or via a extended-release IUD.

In some embodiments the disclosure provides the inhibitor for use as described herein, wherein the female mammalian subject is a human.

In some embodiments the disclosure provides an intra-uterine device comprising a non-steroidal angiogenesis inhibitor.

In some embodiments the disclosure provides the intra-uterine device as described herein, wherein the intra-uterine device further comprises progesterone or progestin.

In some embodiments the disclosure provides the inhibitor for use or the intra-uterine device as described herein, where in the angiogenesis inhibitor is a small molecule or an antigen binding molecule such as an antibody or antigen binding fragment thereof.

In some embodiments the disclosure provides the inhibitor for use or the intra-uterine device as described herein, wherein the angiogenesis inhibitor targets an angiogenic signaling axis, e.g. VEGF, VEGF receptor, EGF, EGF receptor, PDGF, PDGF receptor, or PGF, PGF receptor.

In some embodiments the disclosure provides the inhibitor for use or the intra-uterine device as described herein, wherein the angiogenesis inhibitor targets an angiogenesis-associated molecule or receptor, e.g. an integrin, CD36, CD44, extracellular vimentin, fibrillin-2, secreted frizzled-related protein-2, lysyl oxidase, prostate specific membrane antigen, versican, apelin.

In some embodiments the disclosure provides the inhibitor for use or the intra-uterine device as described herein, wherein the angiogenesis inhibitor is an anti-VEGF binding molecule such as an antibody or antigen binding fragment thereof, a peptibody or a nanobody.

In some embodiments the disclosure provides the inhibitor for use or the intra-uterine device as described herein, wherein the angiogenesis inhibitor is selected from bevacizumab, ramucirumab, or ranibizumab.

In some embodiments the disclosure provides the inhibitor for use or the intra-uterine device as described herein, wherein the small molecule is a tyrosine kinase inhibitor, preferably selected from axitinib, imatinib, erlotinib, cabozantinib, lapatinib, pazopanib, ponatinib, regorafenib, sunitinib, sorafenib, vandetanib, and crizotinib and/or a pharmaceutically acceptable salt thereof.

### BRIEF DESCRIPTION OF THE DRAWINGS

FIG. 1: Schematic overview of experimental groups and timeline for inducing adenomyosis in CD1 mice using tamoxifen, followed by treatment with axitinib 25 mg/kg (dose I) or axitinib 3 mg/kg (dose II) from week 6 until week 9. After termination of the mice at week 9, all uteri were analyzed for the presence of adenomyosis.

FIG. 2: Microscopic images of H&E-stained mouse uteri from this experiment. Classification of depth of ectopic endometrium infiltration in adenomyosis in a mouse model with induced adenomyosis. The arrows show where the ectopic endometrium invades the myometrium.

FIG. 3: Representative images of HE (top row) and α-SMA (bottom row) stained transverse sections of mice uteri after neonatal treatment with vehicle (left column) and tamoxifen (right column). The uteri of vehicle treated mice show no signs of ectopic endometrium in the myometrium (Grade 0) on the HE stained slice, and an intact myometrium on the α-SMA stained slice, while the uteri of tamoxifen treated mice demonstrate ectopic endometrium (black arrow) in the myometrium (Grade 2 presented) on the HE stained slice, and an interrupted myometrium on the α-SMA stained slice with thin residual unaffected myometrium.

FIG. 4: Top: From left to right the HE, α-SMA and Vimentin staining results of grade I adenomyosis in transverse sections of mice uteri. Bottom: from left to right the HE, α-SMA and Vimentin staining results of grade II adenomyosis in transverse sections of mice uteri. Black arrow indicates the invaded ectopic endometrium with thin residual unaffected myometrium in each uterine transverse section.

FIG. 5: Top: Bar chart of mice (count) that presented with Grade 0/1/2/3 of adenomyosis per treatment group; CMC; Placebo, AX3; Dose II axitinib 3 mg/kg, AX25; Dose I axitinib 25 mg/kg. Bottom: Bar chart of mice (percentages) that presented with Grade 0/1 or 2/3 of adenomyosis per treatment group; placebo (CMC, n=34) vs. Axitinib (3 mg/kg or 25 mg/kg) showing that mice treated with axitinib had a lower incidence of Grade 2/3 adenomyosis than mice treated with placebo (p=0.048).

FIG. 6: Timeline for determining stage of adenomyosis at 4 weeks.

FIG. 7: Timeline for intra-uterine administration of axitinib; a) short-term and b) long-term experiments.

FIG. 8: Timeline for fertility experiments.

FIG. 9: Bar chart showing adenomyosis severity index (determined as mean grade of adenomyosis×mean percentage surface area of adenomyosis per mouse uterus specimen). Data are shown as median f range for all mice pooled. * significance p<0.05, ** p<0.001. Treatment groups: placebo (CMC, n=34), AX3 (dose II: axitinib 3 mg/kg, n=33) and AX25 (dose I: axitinib 25 mg/kg, n=34).

FIG. 10: Real-time (RT)-PCR molecular profiling of the mouse uteri treated with CMC, axitinib dose II (AX3=3 mg/kg), or axitinib dose I (AX25=25 mg/kg). Expression fold change of angiogenesis-related genes plGF, VEGF-R1, VEGF-R2, VEGF-R3, VEGF-A, VEGF-B, CD31 and Collagen determined by 2\hat{ }-ΔΔCt of RT-PCR. Relative gene expressions are shown as mean±SEM. CMC n=7; AX3 (dose II) n=6; AX25 (dose I) n=7. *p<0.05 and **p<0.001.

### DETAILED DESCRIPTION OF THE DISCLOSED EMBODIMENTS

While not wishing to be bound by theory, we hypothesized that angiogenesis plays a pivotal role in the development of adenomyosis and other uterine disorders and that the inhibition of this process could result in suppressed myometrial infiltration by endometrial tissue and alleviation of symptoms. Accordingly, the disclosure provides angiogenesis inhibitors for treating or preventing benign uterine disorders.

The disclosure provides methods for treating or preventing a benign uterine disorder, e.g. adenomyosis, in a female mammalian subject. In particular the methods are for a reduction of the severity of the benign uterine disorder and/or a reduction of the symptoms associated with the benign uterine disorder. The method comprises locally administering to a subject in need thereof a non-steroidal angiogenesis inhibitor.

The disclosure provides a non-steroidal angiogenesis inhibitor in a method for treating or preventing a benign uterine disorder in a female mammalian subject, comprising locally administering to a subject in need thereof the non-steroidal angiogenesis inhibitor.

The term “treating” as used herein refers to a reduction of the severity of a benign uterine disorder, and/or a reduction of the growth of the endometrial tissue into the muscular wall of the uterus and/or reduction of one or more of the symptoms associated with a benign uterine disorder and/or restoration of fertility. Preferably, said treatments result in restoration or improvement of the health of a subject or individual. Preferably, the subject or individual has less disease symptoms or experiences symptoms for a shorter time. Preferably, said treatments result in restoring fertility, increasing fertility, increasing pregnancy rates, and/or reducing risk of miscarriage.

The term “preventing” as used herein refers to the prevention of development of a benign uterine disorder and/or prevention of growth of the endometrial tissue into the muscular wall of the uterus and/or prevention of one or more of the symptoms associated with a uterine being disorder and/or prevention against loss of fertility. As recognized by a skilled person, prevention does not mean that none of the treated females will ever develop said disorder. Rather, prevention refers to a decreased likelihood that a female with develop said disorder or symptoms as compared to a non-treated female.

In some embodiments the method preserves fertility. Administering the non-steroidal angiogenesis inhibitor reduces the benign uterine disorder or prevents the development of the benign uterine disorder. This can prevent damage to the uterus and associated tissues and thus preserve the healthy function and the fertility of the female mammalian subject.

The term “benign uterine disorder” refers to a condition that affects the uterus, specifically referring to inside the uterus. These conditions are benign. Benign refers to a condition, tumor, or growth that is not cancerous. This means that it does not spread to other parts of the body. It does not invade nearby tissue. Examples of diseases that affect the uterus are adenomyosis, benign tumors, polyps, fibroids, Cesarean scar defects (niche) and Menometrorrhagia (abnormal uterine bleeding). Preferably the benign uterine disorder is selected from benign uterine tumors, uterine polyps, uterine fibroids and adenomyosis. The term “benign uterine disorder” does not include diseases outside the uterus, e.g. endometriosis.

Benign uterine disorders can cause various symptoms such as abnormal uterine bleeding (AUB), e.g. heavy or prolonged menstrual bleeding, severe cramping or sharp, knifelike pelvic pain during menstruation (dysmenorrhea), pain, chronic pelvic pain, painful intercourse (dyspareunia), subfertility (reduced fertility/infertility). AUB is defined by the International Federation of Gynecology and Obstetrics (FIGO) as a set of different menstrual symptoms caused by several uterine abnormalities, most often presenting as intermenstrual or heavy menstrual bleeding (IMB and HMB, respectively). AUB can be diagnosed if one or more of the following symptoms occur; 1) the patients menstrual frequency, duration, regularity and/or flow volumes are abnormal according to FIGO AUB definitions. 2) The patient experiences IMB, defined as bleeding between cyclically regular onsets of menstruation, and 3) the patient has unscheduled bleeding on medication with progestin, with or without oestrogens such as birth control pills, intra-uterine devices, injections etc. (Munro, M. G., H. O. D. Critchley, I. S. Fraser and F. M. D. Committee (2018). “The two FIGO systems for normal and abnormal uterine bleeding symptoms and classification of causes of abnormal uterine bleeding in the reproductive years: 2018 revisions.” Int J Gynaecol Obstet 143(3): 393-408). When spotting is caused by medical interventions or devices (e.g., oestrogen or progestin therapy via systemic or intrauterine delivery routes) it is classified as iatrogenic-AUB (AUB-I). In some cases, the benign uterine disorder does not cause symptoms or only mild symptoms.

In some embodiments the benign uterine disorder is adenomyosis. Adenomyosis is a benign condition where endometrium tissue is present in the myometrium layer of the uterus. Adenomyosis occurs when the endometrial tissue that normally lines the uterus grows into the muscular wall of the uterus. The displaced tissue continues to act normally during each menstrual cycle, including thickening, breaking down and bleeding. As a result, the uterus can be enlarged and painful, heavy periods can occur.

Sometimes, adenomyosis causes no signs or symptoms or only mild discomfort. However, adenomyosis can cause: heavy or prolonged menstrual bleeding, severe cramping or sharp, knifelike pelvic pain during menstruation (dysmenorrhea), chronic pelvic pain, painful intercourse (dyspareunia). In some cases, the uterus is enlarged. An enlarged uterus may result in tenderness or pressure in the lower abdomen. Known symptoms of adenomyosis include painful menstrual cramps (dysmenorrhea), heavy menstrual bleeding (menorrhagia), abnormal menstruation, pelvic pain, painful intercourse (dyspareunia), subfertility (reduced fertility), infertility, increased miscarriage rate and an enlarged uterus. The symptoms have a considerable impact on quality of life.

In the current medical practice, the symptoms of adenomyosis can be treated by suppressing the menstrual cycle with hormonal therapy. However, many women experience too many side effects and no full relief of symptoms with this therapy, and have to turn to more radical therapy such as embolization (only available in research setting) or hysterectomy (Pontis, D'Alterio et al. 2016), both are incompatible with a child wish or restoring fertility.

As recognized by a skilled person, adenomyosis is a different disease than endometriosis. In contrast to adenomyosis, endometriosis is a disorder in which the endometrium grows outside the uterus. Endometriosis most commonly involves the ovaries, fallopian tubes and the tissue lining the pelvis.

With endometriosis, the endometrial-like tissue outside the uterus acts as endometrial tissue would. With each menstrual cycle the tissue thickens, breaks down and bleeds. Because this tissue has no way to exit the body, it becomes trapped in the abdomen. When endometriosis involves the ovaries, endometriomas may form. The surrounding tissue can become irritated, eventually developing scar tissue and adhesions. Endometriosis can cause pain especially during menstrual periods. Fertility problems also may develop.

The term “female mammalian subject” refers to any female mammalian. Adenomyosis and other benign uterine disorders are diseases that develop in the uterus, therefore this disease can affect female mammalians. In preferred embodiments, the female mammalian subject is a woman. In preferred embodiments, the female mammalian subject is post-menarche. Menarche is the first menstrual cycle, or first menstrual bleeding, in female humans. In preferred embodiments, the female mammalian subject is post-menarche and pre-menopausal. Preferably, the female mammalian subject is in her “fertile” years, and/or child bearing age. The female mammalian subject, may be multiparous or nulliparous. Multiparous women have given birth one or more times before in the past, while nulliparous women have never having given birth before.

The method of the present invention comprises administering the composition described herein to a human individual in need thereof. In some embodiments, the individual is afflicted with a benign uterine disorder.

Most cases of adenomyosis are found in women in their 40s and 50s. Adenomyosis in these women could relate to longer exposure to estrogen compared with that of younger women. However, current research suggests that the condition might also be common in younger women. Risk factors for adenomyosis include: multiparity (childbirth), previous abortion, prior uterine surgery, such as C-section, fibroid removal, or dilatation and curettage (D&C). Furthermore, women of middle age and/or women having irregular cycles are more risk of having adenomyosis

Whereas in the past this condition was thought to mainly develop in multiparous women, improved diagnostic methods have shown that it is also present in nulliparous women and it is associated with a 28% lower clinical pregnancy rate as well as a more than doubled risk of miscarriage in women undergoing IVF (Martinez-Conejero, Morgan et al. 2011, Tremellen and Russell 2011, Vercellini, Consonni et al. 2014).

In the present disclosure the non-steroidal angiogenesis inhibitor is administered locally. The term “locally administering” can refer to intrauterine administration and/or intra-myometrial administration. Intra-myometrial administration refers to administration of the composition within the muscular coat of the uterus. The preferred administration is intrauterine administration. The term “intrauterine administration” refers to administration within the uterus.

Local administration or local therapy in general has a more favorable risk profile since it can reach equivalent effectiveness to systemic therapy, without a significant rise in serum drug concentrations. In contrast to systemic administration, local administration requires lower doses and/or amounts, can reduce possible side effects of the angiogenesis inhibitors and increase treatment effectivity. Further, local administration of the non-steroidal angiogenesis inhibitor can reduce benign uterine disorders, angiogenesis, adenomyosis-related AUB and/or fertility difficulties without disrupting physiological angiogenesis in the body, organs and tissues and/or reproductive organs. Some other advantages of local administration can include, but are not limited to, immediate onset of action, possible prolonged action, avoiding the gastro-intestinal incompatibility and high levels of patient compliance and satisfaction, e.g. no issue of unpleasant taste and smell as can be experienced with oral intake.

The term “angiogenesis” refers to the physiological process through which new blood vessels form from pre-existing vessels. Angiogenesis continues the growth of the vasculature by processes of sprouting and splitting. It is a sophisticated process, regulated by the balance between endogenous, pro-angiogenic (or stimulatory) and anti-angiogenic (or inhibitory) factors.

Upon the activation by one or more pro-angiogenic factors, endothelial cells resting in the parent vessels synthesize and release degrading enzymes allowing the endothelial cells to migrate, proliferate and finally differentiate to give rise to blood vessels. Angiogenesis is a normal and vital process in growth and development. Angiogenesis normally takes place during embryonic and fetal organogenesis, reproductive cycle (e.g., female menstrual cycle), repair processes, wound healing processes and tissue regeneration. However, in many pathological conditions, the disease appears to be associated with upregulated angiogenesis.

An angiogenesis inhibitor is a substance that inhibits the growth of new blood vessels (angiogenesis). Angiogenesis inhibitors have been closely studied for possible cancer treatments. Studies on cancer usually use the highest possible dose, to reach the highest effect. “Bombing” the tumor with angiogenesis inhibitors may be acting more as chemotherapy, masking the specific anti-angiogenesis effect. Furthermore, malignant cells have evolved an angiogenic response that is different from physiological angiogenesis, which is still responsive to intervention. Additionally, tumor cells are genetically instable and keep mutating towards therapy-resistant variants. As endometrial cells are benign and genetically stable, therapy resistance is less likely to occur. It is for these reasons that anti-angiogenic intervention in benign uterine disorders is expected to be successful and may lead to the development of an efficient non-hormonal local treatment.

The method of the present disclosure comprises administering of a non-steroidal angiogenesis inhibitor. A non-steroidal compound is a drug that is not a steroid nor a steroid derivative. Suitable angiogenesis inhibitors are known to the skilled person.

While not wishing to be bound by theory, the present disclosure proposes that angiogenesis might play a key etiological factor in the development of adenomyosis. Angiogenesis is the arising of new capillaries out of pre-existing blood vessels and takes place in both physiological and pathological processes (Folkman 1995, Griffioen and Molema 2000). In the menstrual cycle, angiogenesis occurs physiologically during the proliferative or follicular phase, when regeneration of the endometrium occurs (Critchley, Maybin et al. 2020). It is suggested that increased angiogenesis is one of the underlying mechanisms in the pathophysiology of adenomyosis (Li, Chen et al. 2013, Huang, Chen et al. 2014, Liu, Shen et al. 2016, Wang, Deng et al. 2016, Vannuccini, Tosti et al. 2017, Harmsen, Wong et al. 2019), since in adenomyosis, the endometrial cells seem to need angiogenesis to invade and establish at an ectopic location (Kang, Li et al. 2010, Yen, Huang et al. 2017). A study investigating the level of angiogenesis in endometrial tissue retrieved from adenomyosis patients and control patients has shown higher mean vascular density adenomyosis patients during the proliferative phase (Harmsen, Arduc et al. 2022).

While not wishing to be bound by theory, estrogen and progesterone are known angiogenic factors in the uterus, implicating the hormonal role for angiogenesis in adenomyosis patients (Hyder et al., 2000). Estrogen is known to induce EMT—the transition of endometrial epithelial cells (associated with a decrease in E-cadherin) to mesenchymal cells, accompanied by an increase in vimentin (Chen et al., 2010; Ribatti, 2017). This process is linked to angiogenesis, and the same factors that are involved in EMT may drive endothelial cells toward a pro-angiogenic state in adenomyosis (Chen et al., 2010; Khan et al., 2015; An et al., 2017)

While not wishing to be bound by theory, it is proposed herein that treatment with an angiogenesis inhibitor reduces the growth of endometrial tissue in the muscular wall of the uterus, resulting in less adenomyosis and/or the corresponding symptoms. The present disclosure proposes that the therapeutic compounds disclosed herein affect the angiogenesis of the endometrial tissue. Inhibition of the angiogenesis would result in suppressed myometrial infiltration by endometrial tissue and thus alleviation of symptoms. Thus, the present disclosure proposes that the therapeutic compounds disclosed herein target the origin of adenomyosis.

In some embodiments, the female mammalian subject is afflicted with a benign uterine disorder and the method reduces symptoms and/or increases clinical pregnancy rate and live birth rate. Treatment of the female mammalian subject with the method as disclosed herein can reduce the severity of the symptoms associated with benign uterine disorders, for example adenomyosis. It can reduce the clinical symptoms as described above and restore the health of the uterus in the female mammalian subject.

Benign uterine disorders, e.g. adenomyosis can lead to reduced fertility, with a considerable impact on women's quality of life and subsequently on society. It is knowns that the reduced fertility rate leads to a lower clinical pregnancy rate and a greater risk of miscarriage in woman undergoing IVF. The treatment method as disclosed herein can also reduce fertility problems in a female subject. Increasing the fertility results in in increased clinical pregnancy rate and live birth rate.

In some embodiments the method preserves fertility. Administering the non-steroidal angiogenesis inhibitor reduces the benign uterine disorder or prevents the development of the benign uterine disorder. This can prevent damage to the uterus and associated tissues and thus preserve the healthy function and the fertility of the female mammalian subject.

While not wishing to be bound by theory, blocking angiogenesis halts follicular and luteal microvasculature development in the ovary, suppressing ovarian function and progesterone secretion. When applied temporarily, angiogenesis inhibition may treat a uterus affected by a benign uterine disorder, which can be followed by a non-therapeutic period, allowing follicle growth, conception and gestation. In some embodiments, the treatment with non-steroidal angiogenesis inhibitor is temporary. The treatment results in restoring the function and/or health of the uterus.

In some embodiments, the non-steroidal angiogenesis inhibitor is administered intra-uterine. The term “intrauterine administration” refers to administration within the uterus. The uterus is located within the pelvic region immediately behind and almost overlying the bladder, and in front of the sigmoid colon. The human uterus is pear-shaped and about 7.6 cm (3.0 in) long, 4.5 cm (1.8 in) broad (side to side), and 3.0 cm (1.2 in) thick. A typical adult uterus weighs about 60 grams. The uterus can be divided anatomically into four regions: the fundus, the corpus (body), the cervix, and the cervical canal. The cervix protrudes into the vagina.

Preferably, the non-steroidal angiogenesis inhibitor is provided as an extended-release formulation or via an extended-release IUD. Extended release can be achieved by various means known in the art, e.g., reservoir or matrix systems.

In some embodiments, the intrauterine administration is provided by an intrauterine device (IUD). The IUD may slowly release the angiogenesis inhibitor. Preferably, the IUD releases the inhibitor over the course of several months, preferably over the course of several years. In this way the inhibitor is provided for slow/extended release.

The intrauterine device is for example a small, flexible, often T-shaped plastic (polyethylene or polypropylene) device that is inserted into the uterus (such as commercially available Mirena®, Kyleena®, Paragard®). The size of the intrauterine device is usually between 20 and 40 mm. The intrauterine device can also be in the loop shape (such as first generation IUDs, for example Lippes loop), in the shape of number 7 (such as Copper-7/Gravigard), V- or Y-shaped (such as VeraCept) or frameless (such as fourth generation IUDs, for example GyneFix®, FibroPlant®).

Intrauterine devices exist in different forms. The first generation of IUDs is characterized by comprising loops. The second generation is characterized by containing copper (e.g., Paragard®). The third generation is characterized by containing progesterone and/or other hormones. The fourth generation is characterized by being frameless.

In some embodiments, the intrauterine device may contain a reservoir and/or carrier loaded with the non-steroidal angiogenesis inhibitor. A reservoir and/or carrier may comprise a core of suitable polymeric matric impregnated with the non-steroidal angiogenesis inhibitor and/or enveloped/sleeved by a permeable membrane for controlling slow-release of the non-steroidal angiogenesis inhibitor into the uterus over a prolonged time. Suitable IUDs for providing therapeutics to the uterine are well-known to the skilled person and are also described in e.g., U.S. Pat. Nos. 4,284,074, 8,118,028, 9,180,039 (which are incorporated by reference). See also Bao et al. Int J Pharm. 2018 Oct. 25; 550(1-2): 447-454.

In some embodiments, the intrauterine device is at least partially coated with the non-steroidal angiogenesis inhibitor.

In some embodiments, the intrauterine device comprises an extended-release composition containing the non-steroidal angiogenesis inhibitor. Preferred dosage forms can be used to provide extended-release of the angiogenesis inhibitor using, for example, hydropropylmethyl cellulose, other polymer matrices, gels, permeable membranes, osmotic systems, multilayer coatings, microparticles, liposomes, microspheres, or a combination thereof to provide the desired release profile in varying proportions.

In some embodiments, the angiogenesis inhibitor is a small molecule or an antigen binding molecule such as an antibody or antigen binding domain thereof.

In some embodiments, the angiogenesis inhibitor is a small molecule. As used herein, the term “small molecule” refers to molecules that bind specific biological macromolecules and act as an effector, altering the activity or function of the target. Small molecules can have a variety of biological functions or applications, serving as cell signaling molecules, drugs in medicine, pesticides in farming, and in many other roles. These compounds can be natural (such as secondary metabolites) or artificial (such as antiviral drugs); they may have a beneficial effect against a disease (such as drugs)

In some embodiments, the inhibitors described herein are antigen binding molecules. Preferred antigen binding molecules are antibodies. As used herein, the term “antibody” includes, for example, both naturally occurring and non-naturally occurring antibodies, polyclonal and monoclonal antibodies, chimeric antibodies and wholly synthetic antibodies and fragments thereof, such as, for example, the Fab′, F(ab′)2, Fv or Fab fragments, or other antigen recognizing immunoglobulin fragments. Methods of making antibodies are well known in the art and many suitable antibodies are commercially available. Preferably, the antibodies disclosed herein include antigen binding fragments (e.g., Fab′, F(ab′)2, Fv or Fab fragments). Preferred antibodies or antigen-binding fragments thereof are humanized or human antibodies or antigen-binding fragments thereof. In some embodiments, the angiogenesis inhibitor is an antigen binding domain of an antibody. In some embodiment the antigen binding domain consists of two binding domains that are linked together.

Suitable antigen binding molecules also include peptibodies. The term “peptibody” as disclosed herein refers to a fusion of a peptide to a part or all of an antibody. A peptibody is composed of two moieties, a biologically active peptide and an Fc region. By fusing a peptide to part or all of an antibody, a peptibody combines the activity of a peptide with the longer duration of activity of an antibody.

Suitable antigen binding molecules also include nanobodies. The term “nanobody” as disclosed herein refers to a single-domain antibody fragment (sdAb, called Nanobody by Ablynx, the developer). This is an antibody fragment with a single monomeric variable antibody region. Like a whole antibody, it is able to bind selectively to a specific antigen. With a molecular weight of only 12-15 kDa, single-domain antibody fragments are much smaller than common antibodies (150-160 kDa) which are composed of two heavy protein chains and two light chains, and even smaller than Fab fragments (˜50 kDa, one light chain and half a heavy chain) and single-chain variable fragments (˜25 kDa, two variable regions, one from a light and one from a heavy chain). Single-domain antibodies by themselves are not much smaller than normal antibodies (being typically 90-100 kDa). Single-domain antibody fragments are mostly engineered from heavy-chain antibodies found in camelids; these are called VHH fragments (Nanobodies®).

In some embodiments, the angiogenesis inhibitor targets an angiogenesis signaling axis and/or angiogenesis signaling pathway or component of a angiogenesis signaling pathway component. The angiogenesis axis includes factors and receptors, such as VEGF, VEGF receptor, EGF, EGF receptor, PDGF, PDGF receptor, and PGF, PGF receptor. Targeting these factors and associated receptors will affect the cell signaling. Modulated signaling of these factors can result in reduced angiogenesis. Thus, targeting these factors and associated receptors is a well-known mechanism for angiogenesis inhibition.

Vascular endothelial growth factor (VEGF), originally known as vascular permeability factor (VPF), is a signal protein produced by many cells that stimulates the formation of blood vessels. To be specific, VEGF is a sub-family of growth factors, the platelet-derived growth factor family of cystine-knot growth factors. They are important signaling proteins involved in both vasculogenesis (the de novo formation of the embryonic circulatory system) and angiogenesis (the growth of blood vessels from pre-existing vasculature). All members of the VEGF family stimulate cellular responses by binding to tyrosine kinase receptors (the VEGFRs or VEGF receptor) on the cell surface, causing them to dimerize and become activated through transphosphorylation. Expression of VEGF in endometrial epithelial cells is found to be different in the early, mid and late proliferative phases of the menstrual cycle (Sugino et al. 2002).

Inhibition of the VEGF pathway has become the focus of angiogenesis research, as approximately 60% of malignant tumors express high concentrations of VEGF. Strategies to inhibit the VEGF pathway include antibodies directed against VEGF or VEGFR, soluble VEGFR/VEGFR hybrids, and tyrosine kinase inhibitors. The most widely used VEGF pathway inhibitor on the market today is Bevacizumab.

Bevacizumab binds to VEGF and inhibits it from binding to VEGF receptors. Epidermal growth factor (EGF) is a protein that stimulates cell growth and differentiation by binding to its receptor, EGFR. EGF, via binding to its cognate receptor, results in cellular proliferation, differentiation, and survival.

EGF acts by binding with high affinity to epidermal growth factor receptor (EGFR) on the cell surface. This stimulates ligand-induced dimerization, activating the intrinsic protein-tyrosine kinase activity of the receptor (see the second diagram). The tyrosine kinase activity, in turn, initiates a signal transduction cascade that results in a variety of biochemical changes within the cell that ultimately lead to DNA synthesis and cell proliferation.

Platelet-derived growth factor (PDGF) is a growth factor that regulates cell growth and division. In particular, PDGF plays a significant role in blood vessel formation, the growth of blood vessels from already-existing blood vessel tissue. Platelet-derived growth factor is a dimeric glycoprotein that can be composed of two A subunits (PDGF-AA), two B subunits (PDGF-BB), or one of each (PDGF-AB).

The receptor for PDGF, PDGFR is classified as a receptor tyrosine kinase (RTK), a type of cell surface receptor. Upon activation by PDGF, these receptors dimerise, and are “switched on” by auto-phosphorylation of several sites on their cytosolic domains, which serve to mediate binding of cofactors and subsequently activate signal transduction. Downstream effects of this include regulation of gene expression and the cell cycle.

Placental growth factor (PGF) is a member of the VEGF (vascular endothelial growth factor) sub-family. PGF is also known as PIGF. PGF is a key molecule in angiogenesis and vasculogenesis, in particular during embryogenesis. The main source of PGF during pregnancy is the placental trophoblast. PGF is also expressed in many other tissues, including the villous trophoblast. PGF is ultimately associated with angiogenesis. Specifically, PGF plays a role in trophoblast growth and differentiation.

Inhibiting angiogenesis requires treatment with anti-angiogenic factors, or drugs which reduce the production of pro-angiogenic factors, prevent them binding to their receptors or block their actions. The factors and associated receptors as described above can be targeted by angiogenesis inhibitors, such as small molecules, antibodies or an antigen binding domain thereof. Targeting these factors and associated receptors will affect the cell signaling. Modulated signaling of these factors can result is reduced angiogenesis. Thus, targeting these factors and associated receptors is a well-known mechanism for angiogenesis inhibition.

In some embodiments the angiogenesis inhibitor targets an angiogenesis-associated molecule or receptor. Angiogenesis-associated molecules or receptors and molecules or receptors that play a role in angiogenesis. These molecules can for example regulate the onset of angiogenesis or the cell signaling associated with the formulation of new blood vessels. The following molecules and receptors are non-limiting examples that are involved in angiogenesis. Integrin, CD36, CD44, extracellular vimentin, fibrillin-2, secreted frizzled-related protein-2, lysyl oxidase, prostate specific membrane antigen, versican, apelin, α-SMA, activin A, COX-2, MMP-2, MMP-9, DJ-I.

Integrin is a heterodimeric transmembrane cell adhesion molecule that mediates cell-cell and cell-extracellular matrix adhesion, and signal transduction between extracellular and intracellular environment. Integrin is composed of a single α and a single β subunit. Through different combinations of the α and β subunits, over 24 mammalian integrins are known so far. Integrins regulate cellular growth, proliferation, migration, signaling, and cytokine activation and release. Thus, integrins play important role in cell proliferation and migration, apoptosis, tissue repair, as well as in all processes critical to inflammation, infection, and angiogenesis. Particularly, integrin and VEGF interact closely in several intracellular angiogenic signaling. Moreover, integrins play critical role in development and vasculogenesis.

Cluster of differentiation 36 (CD36) is a protein that acts as a transporter and sensor of free fatty acids, and a scavenger receptor that binds a number of factors and participates in the internalization of cells, pathogens, and various lipoproteins. CD36 plays a role in the regulation of angiogenesis. Specifically, following binding of the angiogenic inhibitor (e.g. thrombospondin 1, TSP-1), CD36 initiates anti-angiogenic signals, thereby inducing apoptosis or blocking the VEGFR pathway.

Cluster of differentiation 44 (CD44) is a s a cell-surface glycoprotein involved in cell-cell interactions, cell adhesion and migration. Further, CD44 was shown to act as a co-receptor for the receptor tyrosine kinase. CD44, particularly isoform CD44v6, can recruit ezrin/radixin/moesin (ERM) proteins that promote cytoskeletal changes and that may interact with VEGFR contributing to angiogenesis.

Extracellular vimentin is a type III intermediate filament protein that is expressed in endothelial and other mesenchymal cells. Vimentin plays important role in cell adhesion, migration, angiogenesis, neurite extension, and cancer. Phosphorylation of vimentin's specific residues regulates its assembly and involvement in migration and invasion, as well as reorganization in mitosis. Specifically, vimentin has been shown to regulate ligand-specific Notch signaling that regulates angiogenesis. Lack of vimentin is associated with defects in vascular tuning, endothelial migration, adhesion, and sprouting, as well as flow-induced arterial remodeling.

Fibrillin-2 is a glycoprotein found in extracellular matrix. Fibrillin-2, together with other proteins, forms threadlike elastic filaments called microfibrils that enable the skin, ligaments and blood vessels to stretch. Fibrillin-containing microfibrils play a key role in matrix deposition, storage and activation of TGF-β, which is known to play an important role in the regulation of angiogenesis. Interestingly, fibrillin-2 has a lower affinity to TGF-β compared to fibrillin-1, leading to a locally higher active TGF-β concentration.

Secreted frizzled-related protein-2 (sFRP-2) is a glycoprotein containing a so-called frizzled-like cysteine-rich domain. sFRP-2 is known to regulate Wnt signaling which appears to be essential in vascular endothelial cells. sFRP-2 is pro-angiogenic and was shown to increase endothelial cell migration and tube formation.

Lysyl oxidase (LOX) is a copper-dependent amine oxidase that covalently cross-links collagen and elastin in the extracellular matrix (a collagen cross-linking enzyme). LOX has a major role in remodelling the vascular extracellular matrix during angiogenesis. Furthermore, it has been shown that LOX gene expression is highly downregulated in eutopic endometrium of women with versus without adenomyosis.

Prostate specific membrane antigen (PSMA, also known as NAAG peptidase or FOLH1) is an enzyme that catalyzes the hydrolysis of N-acetylaspartylglutamate (NAAG) to glutamate and N-acetylaspartate (NAA). PSMA is upregulated in a wide variety of tumors and inflammatory diseases, likely associated with angiogenesis. Specifically, it has been shown that PSMA inhibition, knockdown, or deficiency decreases endothelial cell invasion in vitro via integrin and PAK, thus abrogating angiogenesis.

Versican is a large extracellular matrix proteoglycan that is found in the extracellular matrix of most soft tissues. Versican plays a central role in tissue morphogenesis and maintenance through cell adhesion, migration, proliferation and angiogenesis. VCAN is one of the molecules upregulated in endometriosis, while its expression is downregulated in adenomyosis.

Apelin is an endogenous ligand for the G-protein-coupled apelin (APJ) receptor.

Apelin is involved in a variety of physiological processes such as vasoconstriction and dilation, the control of blood pressure and blood flow, strengthening of cardiac contractility, angiogenesis, and modulation of energy metabolism and fluid homeostasis. Activation of the apelin/APJ pathway promotes angiogenesis.

In some embodiments the angiogenesis inhibitor is a molecule that binds VEGF, preferably the inhibitor binds VEGF and blocks binding and/or signaling with the VEGF receptor. Preferably the inhibitor is an anti-VEGF antibody or antibody fragment thereof, peptibody or nanobody. In preferred embodiment the antibody is selected from bevacizumab, ramucirumab, or ranibizumab.

An anti-VEGF antibody refers to an antibody that can bind to VEGF e.g. bevacizumab and ranibizumab.

Bevacizumab is a monoclonal antibody that functions as an angiogenesis inhibitor. It is a recombinant humanized monoclonal antibody. Becacizumab works by slowing the growth of new blood vessels by inhibiting vascular endothelial growth factor A (VEGF-A), in other words anti-VEGF therapy.

Ramucirumab is a direct VEGFR2 antagonist, that binds with high affinity to the extracellular domain of VEGFR2 and block the binding of natural VEGFR ligands (VEGF-A, VEGF-C and VEGF-D). These ligands are secreted by solid tumors to promote angiogenesis (formation of new blood vessels from pre-existing ones) and enhance tumor blood supply. Binding of ramucirumab to VEGFR2 leads to inhibition of VEGF-mediated tumor angiogenesis.

Ranibizumab is a monoclonal antibody that inhibits angiogenesis by inhibiting vascular endothelial growth factor A, a mechanism similar to that of Bevacizumab.

In some embodiments the small molecule is a tyrosine kinase inhibitor. In preferred embodiments the tyrosine kinase inhibitor is selected from axitinib, imatinib erlotinib, cabozantinib, lapatinib, pazopanib, ponatinib, regorafenib, sunitinib, sorafenib, vandetanib, and crizotinib.

In some embodiments the small molecule is a pharmaceutically acceptable salt of tyrosine kinase inhibitor. Examples of pharmaceutically acceptable salt are mesylate, tosylate, malate, hydrochloride, ditosylate and succinate. For example, Gleevec is a pharmaceutically acceptable salt of a tyrosine kinase inhibitor.

Axitinib selectively inhibits vascular endothelial growth factor receptors (VEGFR-1, VEGFR-2, VEGFR-3). Through this mechanism of action, axitinib blocks angiogenesis. In the clinic this medication is currently used to inhibit tumour growth and metastases. Axitinib is an indazole derivative and is currently available in oral formulations.

Imatinib (Gleevec—imatinib mesylate) is a first generation TKI. Imatinib is a 2-phenylamino pyrimidine-based compound and binds close to the ATP binding site. Imatinib primary targets Bcr-Abl and thus inhibits cell proliferation and induces apoptosis. Other targets of imatinib include c-KIT and PDGFRs. Imatinib is used to treat chronic myeloid leukemia (CML), metastatic malignant gastrointestinal stromal tumors (GIST) and a number of other malignancies

Dasatinib is a second-generation BCR-ABL inhibitor that has 325-fold higher potency in vivo with inhibitory activity against the majority of imatinib-resistant BCR-ABL mutants.

Nilotinib (Tasigna) was rationally designed to have enhanced selectivity and potency toward BCR-ABL, with clinical activity against the majority of resistant mutations to imatinib and was recommended as second line therapy for CP and AP patients resistant or intolerant to a previous imatinib.

Erlotinib is a first-generation selective and reversible EGFR-TKI. Erlotinib binds in a reversible fashion to the ATP binding site of the receptor, inhibiting EGFR autophosphorylation and activation of the downstream signal cascade. Erlotinib is a quinazoline compound.

Gefitinib is a reversible EGFR inhibitor, like erlotinib, which interrupts signaling through the epidermal growth factor receptor (EGFR) in target cells. Gefitinib is a quinazoline compound and is a medication used for certain breast, lung and other cancers and is only effective in cancers with mutated and overactive EGFR.

Sunitinib is a multi-targeted TKI. The targets of sunitinib include all receptors for platelet-derived growth factor (PDGFRs) and vascular endothelial growth factor receptors (VEGFRs). Sunitinib also inhibits other receptors, such as c-KIT (CD117), G-CSF-R (CD114) and FLT3 (CD135). It is less potent and selective than axitinib and sorafenib. Sunitinib is an oxindole-based compound.

Crizotinib is an inhibitor of the RTKs, including Anaplastic lymphoma kinase (ALK), Hepatocyte growth factor receptor (HGFR, c-Met), Recepteur d'Origine Nantais (RON) and ROS1 kinases. Crizotinib competitively blocks the ATP-binding site and thus inhibits the phosphorylation and downstream signal transduction. Crizotinib is currently thought to exert its effects through modulation of the growth, migration, and invasion of malignant cells. Other studies suggest that crizotinib might also act via inhibition of angiogenesis in malignant tumors. Crizotinib is approved for the treatment of non-small cell lung cancers (NSLCL) and lymphomas expressing activating translocations or mutations of oncogenic tyrosine kinases (in particular ALK and ROS1). Crizotinib is an aminopyridine-based compound.

Pazopanib is a second-generation multi-targeted TKI. Pazopanib attenuates angiogenesis via inhibition of receptors including VEGFR, PDGFR, c-KIT and FGFR. Pazopanib has increased selectivity for VEGFR, but retains activity against other targets, such as PDGFR. Pazopanib is an indazole-based compound.

Ponatinib is a second-generation multi-targeted TKI and a third-generation Bcr-Abl inhibitor. Ponatinib binds to the ATP binding site of the receptor. Ponatinib has an improved inhibitory effect against Bcr-Abl forms with mutations that convey resistance to other TKIs, in particular T315I mutation. Specifically, ponatinib has 520 fold higher potency than imatinib. The primary target of ponatinib is Bcr-Abl, but ponatinib also has potent activity against FLT3, FGFR, and VEGFR family kinases, together with c-Kit and PDGFR. Ponatinib is a benzamide-based compound.

Cabozantinib is a multi-targeted TKI. Cabozantinib downregulates the activation of multiple RTKs involved in tumor angiogenesis, invasion, and metastasis, including MET and VEGFR2. Other targets of cabozantinib include KIT, AXL, RET and FLT3. Cabozantinib is a quinoline-based compound.

Lapatinib is a dual TKI. Lapatinib targets ErbB1/HER1 (EGFR) and ErbB2/HER2 simultaneously. Lapatinib potently and reversibly binds to the ATP-binding pocket inhibits the downstream growth factor signaling pathway (MAPK/PI3K/Akt). Lapatinib is a 4-anilinoquinazoline-based compound.

Sorafenib is a multi-targeted TKI. Sorafenib targets a large variety of kinases, such as RAF, RET, c-KIT, VEGFR and PDGFR, thus reducing angiogenesis and tumor cell proliferation. Sorafenib is a diarylurea-based compound.

Regorafenib is a multi-targeted TKI and is the monofluorinated analog of sorafenib. The introduction of additional fluorine led to a pharmacologically more potent compound with a similar but distinct biochemical profile when compared to sorafenib. Regorafenib targets VEGFRs, FGFR, TIE2, RET, KIT, PDGFR and RAF kinases, which may result in the inhibition of tumor angiogenesis and tumor cell proliferation. Regorafenib is a diarylurea-based compound.

Vandetanib is a multi-targeted TKI. Vandetanib targets EGFRs, VEGFRs, RET, BRK, EPH receptors Tie-2 and Src family members. Vandetanib inhibits various signaling pathways involved in proliferation, growth, invasion/metastasis and angiogenesis. Vandetanib is a 4-anilinoquinazoline-based compound.

The presently existing tyrosine kinase inhibitors are developed over time. The improvement of the molecules has led to different generation of tyrosine kinase inhibitors and will likely yield further generations of molecules in the future. Examples of a first generation tyrosine kinase inhibitors are imatinib, erlotinib, gefitinib and icotinib. Examples of second generation tyrosine kinase inhibitors are: dasatinib, nilotinib, and bosutinib. An example of a third generation tyrosine kinase inhibitor is ponatinib.

In some embodiments the tyrosine kinase inhibitor is a second or third generation tyrosine kinase inhibitor. Second-generation tyrosine kinase inhibitors (TKIs) are more potent drugs and/or bind in an irreversible manner. In cancer therapy the third generation tyrosine kinase inhibitors bind irreversible and have expanded inhibition against a broad spectrum of mutations resistant to first and second generation tyrosine kinase inhibitors.

TNP-70 is a synthetic analog of fumagillin, which binds to and irreversibly inactivates methionine animopeptidase-2 (MetAP2), resulting in endothelial cell cycle arrest late in the G1 phase. An angiogenesis inhibitor as used herein, does not include TNP-70.

The disclosure further provides an intrauterine device (IUD) comprising a non-steroidal angiogenesis inhibitor. The IUD may be loaded and/or at least partially coated with a non-steroidal angiogenesis inhibitor. The intrauterine device can be any of the devices as disclosed in the present application. The non-steroidal angiogenesis inhibitor can ben any of the inhibitors as disclosed in the application.

In some embodiments, the non-steroidal angiogenesis inhibitor is administered in combination with a hormone, in particular progesterone and/or a progestin. The hormone may be administered sequentially or simultaneously with the non-steroidal angiogenesis inhibitor. Preferably, the IUD further comprises a hormone, in particular a progesterone and/or a progestin.

Many women suffer side effects, such as AUB-I, from IUDs comprising a hormone. While not wishing to be bound by theory, an IUD comprising both the hormone and the non-steroidal angiogenesis inhibitor reduces the risk of said side effects and/or treats or reduces the symptoms.

Progesterone belongs to a class of hormones called progestogens. Progesterone is the major and most important progestogen in the body, and is involved in the menstrual cycle, pregnancy and embryogenesis. Progesterone can be used as a medication, such as in the treatment of irregular menstruation, dysmenorrhea, for contraception in combination with or without estrogen, to reduce the risk of uterine or cervical cancer, in hormone replacement therapy, and in feminizing hormone therapy.

Progestins are synthetic progestogens and include preganes (derived from progesterone), estranes and gonanes (both derived from testosterone). Exemplary progestins include ethynodiol diacetate, norethindrone, norethindrone acetate, levonorgestrel, norgestimate, desogestrel, etonogestrel, norelgestromin, gestodene, dienogest, medroxyprogesterone acetate, chlormadinone acetate, cyproterone acetate, drospirenone. Preferred progestins include levonorgestrel and estenogestrel.

The IUD may be loaded and/or at least partially coated with a progesterone and/or a progestin. Preferably, the hormone is provided as an extended-release formulation or via an extended-release IUD. Preferably, the IUD releases the hormone over the course of several months, preferably over the course of several years. In this way the hormone is provided for slow/extended release. IUDs that release both a hormone and a non-steroidal angiogenesis inhibitor have the advantage that the IUD can be used in women as both a contraceptive as well as to treat or prevent a benign uterine disorder. The IUD can then be removed once pregnancy is desired. Without wishing to be bound by theory, the IUD is expected to maintain uterine health thereby increasing fertility once the IUD is removed.

The invention is further explained in the following examples. These examples do not limit the scope of the invention, but merely serve to clarify the invention.

### EXAMPLES

### Example 1

### Methods

### Preparation of Animals

Experiments were approved by the Central Committee for Animal Experiments in the Netherlands. Pregnant CD-1 mice were purchased from Charles River. Each mouse was housed individually during gestation. After giving birth, the dams were housed in one cage with their pups in a controlled environment with 70-80% humidity at 22° C. with a 12 h/12 h light: dark cycle for three weeks. Next, the pups were weaned and allocated to new cages, with a maximum of five animals per cage. Animals were allowed access to chow and water ad libitum. After weaning, the mothers were terminated, the uterus was excised and used as surplus material to optimize immunohistochemical (IHC) staining.

### Mouse Model of Adenomyosis

Adenomyosis was induced in the female mice according to a model previously described (Green, Styles et al. 2005). To validate this model in our laboratory, female mice were dosed with tamoxifen (1 mg/kg; ‘tamoxifen group’; n=6), suspended in peanut oil, by oral gavage, at a dose volume of 5 μl/g bodyweight on day 2-5 after birth, as reported previously (Green, Styles et al. 2005, Zhu, Chen et al. 2016). As control, female mice (n=6) received the peanut oil solvent only (‘vehicle group’; n=6). All mice were sacrificed after six weeks (42 days), to determine the presence and/or degree of adenomyosis.

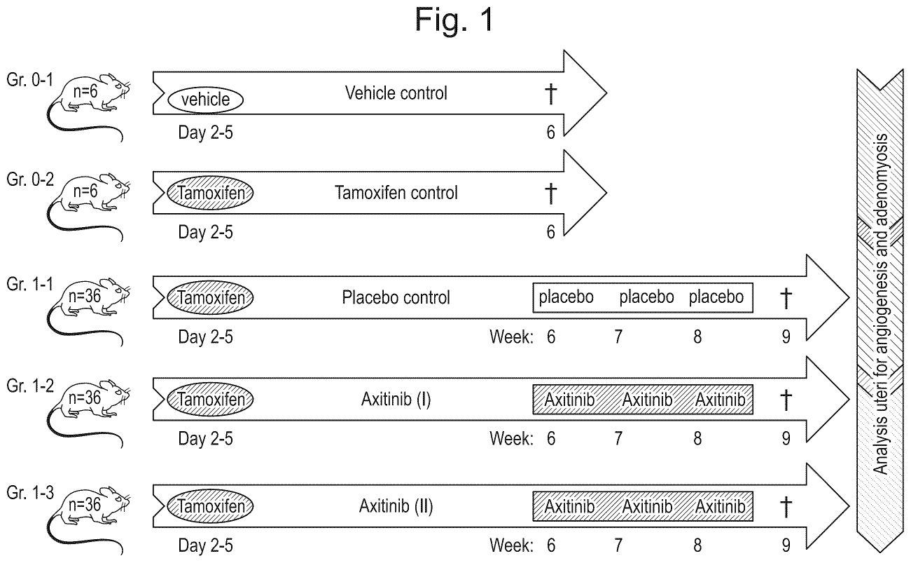

After validation of the model, this model was used to test treatment with anti-vascular endothelial growth factor receptor 2 (VEGFR-2) tyrosine kinase inhibitor axitinib (A-1107; LC Laboratories). To induce adenomyosis, 108 mice received neonatal tamoxifen treatment and were randomly allocated to receive treatment or placebo. On day 42, anti-angiogenesis treatment commenced by oral axitinib treatment or placebo. Axitinib was dissolved in 0.5% sodium carboxymethylcellulose (CMC) at a dose of 25 mg/kg body weight (dose I) or at a dose of 3 mg/kg (dose II). All animals received treatment by daily oral gavage at 5 μl/g bodyweight with axitinib (dose I/II) or placebo (CMC) for a time period of three weeks. During this period, all animals had daily animal welfare observations, and were weighed at a minimum of three times per week. The experimental groups are depicted in FIG. 1.

All mice were sacrificed one day after the last oral gavage day. Blood was drawn, uteri were collected, together with a kidney and liver. The uteri were cut in half, one horn (right/left horn randomized) was fixed in 4% formaldehyde at room temperature for 48 hours. Samples were embedded in paraffin for haematoxylin & eosin (H&E) and IHC staining. The other horn was snap-frozen in liquid nitrogen.

### Immunohistochemistry

Uterine tissue was embedded in paraffin and sliced into transverse oriented sections of 5 μm, with three sections of one uterine horn on one microscope slide. Slides were either stained for H&E or used for IHC. Sections were stained for α-smooth muscle actin (α-SMA) (rabbit polyclonal; cat. no. mAb1420; clone 1A4; 1:200; Novus Biologicals), cluster of differentiation 31 (CD31) (rat monoclonal; cat. no. DIA-310M; clone SZ31; 1:50; Dianova), and vimentin (mouse monoclonal; cat. no. sc-373717; 1:50; Santa Cruz Biotechnology). Paraffin-embedded sections were dewaxed in xylene, rehydrated in 100%, 96%, and 70% ethanol, and washed in PBS (pH 7.4). Antigen retrieval utilizing citrate buffer (pH 6.0) was completed in the autoclave at 98° C. After washing in PBS, blocking of endogenous peroxidase activity was done by 0.3% H2O2/PBS for fifteen minutes. For vimentin staining, blocking of endogenous peroxidase was performed before antigen retrieval in the autoclave. After washing in PBS, aspecific binding was blocked by 3% BSA/PBS for one hour. After adding primary antibodies diluted in 0.5% BSA/PBS, sections were incubated overnight at 4° C. Slides were washed in PBS, subsequently, secondary antibodies were applied for α-SMA (swine anti-rabbit biotinylated antibody; cat. no. ab10935; 1:500; Dako Cytomation), CD31 (donkey anti-rat biotinylated antibody; cat. no. A110-137B; 1:500; Jackson), and vimentin (goat anti-mouse biotinylated antibody; cat. no. ab10721; 1:500; Dako Cytomation) diluted in 0.5% BSA/PBS for 45 min. After washing with PBS, horseradish peroxidase (HRP)—streptavidin (strep) was added (cat. no. ab6734; 1:200; Dako Cytomation) diluted in 0.5% BSA/PBS, for thirty minutes. The sections were then washed in PBS, afterwards 3,3′-Diaminobenzidine (DAB) was added (60 μl DAB; 1 μl 30% H2O2; 1 mL PBS), for eight minutes. Sections were washed in PBS, then counterstained with hematoxylin for thirty seconds. Finally, sections were dehydrated in a mounting absolute alcohol gradient, washed with xylene, and covered with glass coverslips over Quick D mounting medium. Different controls were incorporated by staining a slide without primary antibody, one without primary and secondary antibodies, and one with DAB only.

### Image Analysis

Image capture and analysis were performed using an Olympus BX50 microscope and an image capture device. Digital image analysis was performed using ImageFocus (version 4.0). Microscopic images were examined blind of the histological analysis and original treatment groups. Two-four transverse sections per mouse were analyzed, depending on the quality of the section and/or IHC.

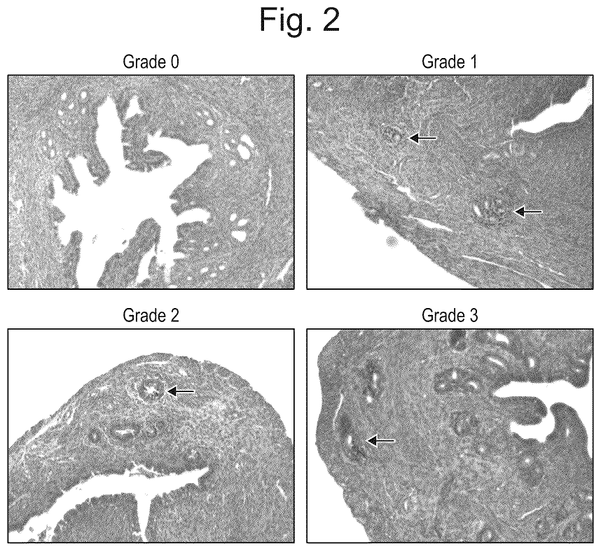

Based on H&E staining, the diagnosis of adenomyosis was established (yes/no) when endometrial glands were present at ectopic sites in the myometrium, as well as the grade of adenomyosis according to the criteria of Bird et al. (Bird, McElin et al. 1972) as reported previously (Jin, Wu et al. 2020). Grade 0 was defined as the total absence of ectopic endometrium in the myometrium. Grade 1 was defined as the penetration of ectopic endometrium into superficial myometrium. Grade 2 suggested a penetration of the ectopic endometrium into the middle of the myometrium, and grade 3 was indicated as a penetration past the mid-myometrium. Examples of the classification of depth of ectopic endometrium infiltration are presented in FIG. 2. The grade was determined based on analyzing two to four uterine tissue sections, where the highest grade, thus the deepest infiltration was the conclusive grade. The number of ectopic glands or ectopic endometrium areas in the myometrium was assessed, the mean number of the analyzed tissue sections was used for further analyses. The presence of adenomyosis was also indicated as ‘focal’ or ‘diffuse’, where focal spread was defined as infiltration of ectopic endometrium in only one quartile of the uterus sample. If the presence of ectopic sides was more scattered, it was described as diffuse. Vimentin staining was used to qualitatively map the endometrium stroma and was previously described in endometrium stroma and blood vessels in mice uteri (Mehasseb, Bell et al. 2009). α-SMA immunostaining was used to quantify parameters of the uterine wall, including the full thickness of the uterine wall (endometrium+myometrium) at thickest point, the full thickness of the uterine wall (endometrium+myometrium) at the thinnest point, the myometrium at the thinnest point (in adenomyosis affected uteri: residual unaffected myometrium at point of maximum ectopic endometrium infiltration), and the myometrium at the thickest point (in adenomyosis affected uteri: hypertrophied myometrium adjacent to myometrium affected by ectopic). Also, assessment of α-SMA staining was used to qualitatively assess the pattern of myometrial smooth muscle cells and intactness or interruption of the inner myometrium. Uterine sections were also examined by experienced pathologists for the presence of fibrosis based on both H&E and phosphotungstic acid haematoxylin (PTAH) staining.

### Statistical Analysis

Statistical analysis was performed using Graphpad Prism software version 9.0 (GraphPad Software, San Diego, California, USA) and SPSS 26.0 software (SPSS, Inc.). Pearson Chi-square tests (for trend) were used to compare categorical variables. When 20% or more of the cells had an expected count less than 5, Fisher's exact test is used. The one-way ANOVA and independent samples T-test for normally distributed data or Mann-Whitney U test for non-normally distributed data were used for comparison of continuous variables between groups. A p-value of ≤0.05 was considered to be statistically significant. Noted range values are minimum−maximum.

### Results

### Validation of Mouse Adenomyosis Model

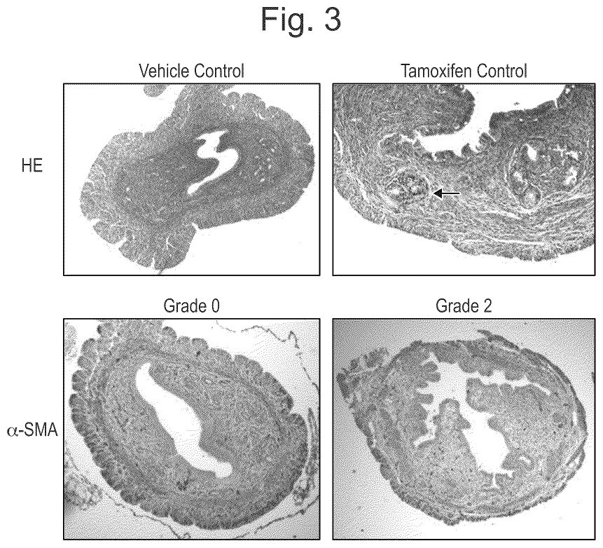

Adenomyosis was successfully induced in 100% of the mice that received tamoxifen for validation of the model (n=6) and was not present in the mice that received vehicle only (n=6), as shown in Table I. All mice that received tamoxifen developed adenomyosis defined as grade 2 (FIG. 3). In the vehicle-treated group, a clearly visible junctional zone was present, with a distinct separation between the endometrium and myometrium visible on HE-stained as well as α-SMA stained uteri. In α-SMA stained uteri, there was loosening and disruption of the circular layer of smooth muscle cells of the inner myometrium in the tamoxifen-treated group, with several glands interrupting and infiltrating the inner myometrium, leading to thin residual unaffected myometrium and hypertrophied adjacent myometrium (FIG. 3). At the thinnest point, the ratio of residual unaffected myometrium to total uterine wall was significantly smaller in the tamoxifen-treated compared to the vehicle-treated group (p<0.05). There was no difference between the groups in the ratio of the thickest point of myometrium to uterine wall, but the ratio of the thinnest point of the myometrium to the thickest point of the myometrium was also smaller in the tamoxifen-treated group (p<0.05). Overall, the results of the α-SMA staining show that the residual unaffected myometrium is thin at the point where endometrium invades the myometrium, while increased myometrium thickness is seen adjacent to the affected sites, which may indicate myometrium hypertrophy, a hallmark of adenomyosis. The lower ratio of the thinnest to the thickest point of myometrium that was seen in the tamoxifen-treated group illustrated this finding (p=0.004) (Table I). Vimentin expression was observed in endometrial stroma and blood vessel walls, in both tamoxifen and vehicle-treated mice. Staining was also observed in ectopic adenomyotic endometrium stroma of mice treated with tamoxifen. No signs of fibrosis surrounding the adenomyosis affected areas in the tamoxifen-treated group were observed by H&E and PTAH staining.

TABLE I
Characteristics of mice in validation experiment.

| | Vehicle | | Tamoxifen | | |
|---|---|---|---|---|---|
| Characteristic | (n = 6) | | (n = 6) | | P-value |
| Weight | | | | | |
| Median | 25.5 | | 25.2 | | 0.936 |
| Range | 22.7-28.8 | | 23.1-29.8 | | |
| Adenomyosis | | | | | |
| No | 6 | (100%) | 0 | (0%) | |
| Yes | 0 | (0%) | 6 | (100%) | |
| Grade | | | | | |
| 0 | 6 | (100%) | 0 | (0%) | |
| 1 | 0 | (0%) | 0 | (0%) | |
| 2 | 0 | (0%) | 6 | (100%) | |
| 3 | 0 | (0%) | 0 | (0%) | |
| Ectopic glands | | | | | |
| Median | 0 | | 7.5 | | |
| Range | 0 | | 3-15 | | |
| Spread | | | | | |
| Focal | 0 | (0%) | 2 | (33.3%) | |
| Diffuse | 0 | (0%) | 4 | (66.7%) | |

| | | | |
|---|---|---|---|
| Uteri parameters (α-SMA) | | | |
| Ratio thinnest myometrium/thinnest uterine wall | 0.58 | 0.18 | 0.004 |
| Ratio thickest myometrium/thickest uterine wall | 0.40 | 0.43 | 1.000 |
| Ratio thinnest myometrium/thickest | 0.65 | 0.19 | 0.004 |
| myometrium | | | |

### Treatment Outcome

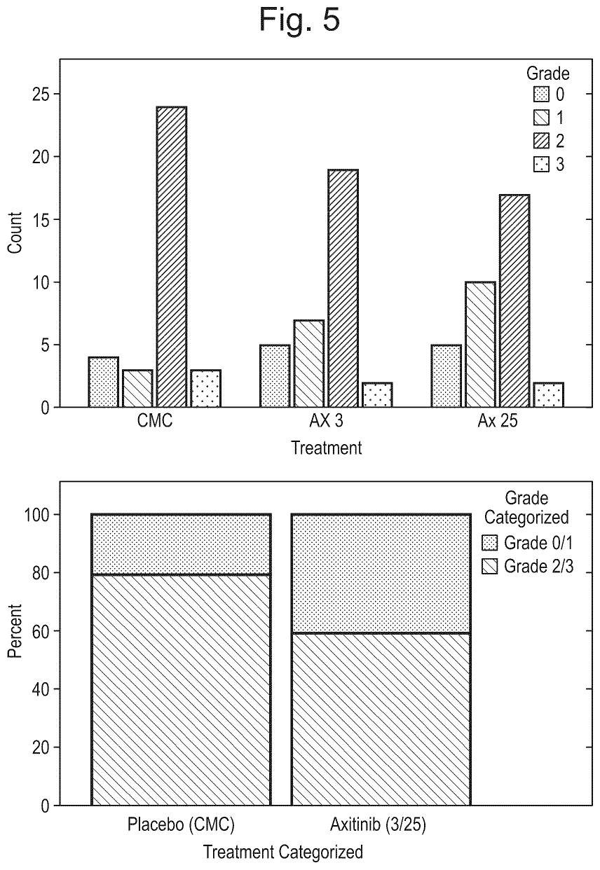

A total of 102 mice completed the neonatal tamoxifen induction and could be analyzed for the primary outcome (Table II). The majority of all mice had developed adenomyosis, without a difference between the treatment groups (p=0.91). In the groups of mice treated with axitinib dose I and dose II, respectively 30 (88,2%) and 28 (82,4%) mice presented with adenomyosis, while in the placebo group, 29 (85,3%) mice presented with adenomyosis (FIG. 5). In four (11,8%) and five (14,7%) of the axitinib dose I and II treated mice, there were no signs of adenomyosis, while five (14,7%) of the placebo mice did not have signs of adenomyosis. Grade 0 and 1 adenomyosis were more prevalent in the groups treated with axitinib dose I/II, while grade 2 and 3 are more prevalent in the placebo group (FIG. 5). There was no significant difference in the number of ectopic glands, or spread of adenomyosis between the groups (Table II). The outcome was also analyzed between the group that was treated with axitinib (dose I+II) and the placebo group and for the outcome grade 0/1 adenomyosis vs. grade 2/3 (Table II). In the pooled data, there is a significant difference between the incidence of grade 0/1 vs grade 2/3 adenomyosis between the group that was treated with axitinib and the group treated with CMC-only (p=0,048; odds ratio 1.33) (Table III, FIG. 5).

TABLE II
Characteristics of mice in anti-angiogenesis treatment experiment.

| | Dose I | | | | | | |
|---|---|---|---|---|---|---|---|
| | Axitinib 25 | | Dose II | | Placebo | | |
| | mg/kg | | Axitinib 3 mg/kg | | CMC-only | | |
| Characteristic | N = 34 | | N = 34 | | N = 34 | | P-value |
| Weight end treatment (g) | | | | | | | |
| Mean | 29.9 | | 28.4 | | 28.9 | | 0.07 |
| SD | 3.1 | | 2.2 | | 2.6 | | |
| Adenomyosis (n, %) | | | | | | | |
| No | 4 | | 5 | | 5 | | 0.91 |
| Yes | 30 | | 28 | | 29 | | |
| Missing | 0 | | 1 | | 0 | | |
| Grade (n, %) | | | | | | | |
| 0 | 5 | (14.7%) | 5 | (15.2%) | 4 | (11.8%) | 0.50 |
| 1 | 10 | (29.4%) | 7 | (21.2%) | 3 | (8.8%) | |
| 2 | 17 | (50.0%) | 19 | (57.6%) | 24 | (70.6%) | |
| 3 | 2 | (5.9%) | 2 | (6.1%) | 3 | (8.8%) | |
| Missing | 0 | | 1 | | 0 | | |
| Ectopic glands, median [Range] | 3.0 | [0.0-11.0] | 2.7 | [0.0-11.8] | 3.3 | [0-27.5] | 0.8 |
| Missing | 1 | | 5 | | 1 | | |
| Spread | | | | | | | |
| None | 5 | (14.7%) | 5 | (15.2%) | 4 | (11.8%) | 0.8 |
| Focal | 8 | | 11 | | 11 | | |
| Diffuse | 18 | | 16.4 | | 18.6 | | |
| Missing | 1 | | 4 | | 0 | | |
| Uteri parameters, median [IQR p25-p75] | | | | | | | |
| Ratio thinnest/thickest myometrium | 0.26 | [0.19-0.40] | 0.23 | [0.20-0.33] | 0.19 | [0.13-0.28] | 0.270 |
| Ratio thinnest myometrium/ | 0.21 | [0.14-0.29] | 0.19 | [0.12-0.25] | 0.16 | [0.13-0.23] | 0.343 |
| uterine wall | | | | | | | |
| Ratio thickest myometrium/uterine | 0.49 | [0.41-0.58] | 0.54 | [0.48-0.61] | 0.55 | [0.51-0.63] | 0.014 |
| wall | | | | | | | |
| Ratio thickest | 0.18 | [0.16-0.20] | 0.18 | [0.15-0.22] | 0.20 | [0.17-0.24] | 0.060 |

myometrium/transverse uterine
diameter

Abbreviations: SD, standard deviation; En, endometrium; M, myometrium.

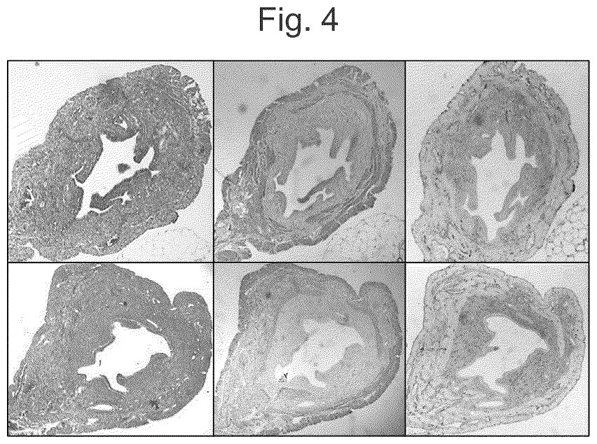

Expression of α-SMA was a clear marker of the longitudinal muscle layer of the myometrium of the mouse uteri. In all tissue sections of both the axitinib as well as the CMC-only treated mice, the junctional zone was interrupted, in accordance with the tamoxifen group of the validation experiment (FIG. 4). At the thickest point of the uterine wall, the ratio of myometrium to total uterine wall was smaller in the placebo group compared to the axitinib-treated groups (K-W p=0.014, post hoc difference between CMC- and AX25 treated group p=0.002) (Table II). In the placebo group compared to the axitinib groups combined, also, the ratio between the thinnest and the thickest point of the myometrium was smaller in the placebo group (p=0.023) (Table II). Overall, the results of the Q-SMA staining in the axitinib versus the placebo treated groups indicate that in the placebo group less residual unaffected myometrium remains at the point where endometrium invades the myometrium, while there is increased myometrium thickness adjacent to the affected sites (higher ratio thickest myometrium/thickest uterine wall), which could be myometrium hypertrophy, a hallmark of adenomyosis. The lower ratio of the thinnest to the thickest point of myometrium that was seen in the placebo-treated group versus the axitinib-treated groups supports this finding.

TABLE III
Outcomes anti-angiogenesis treatment categorized

| | Axitinib | | Placebo | | |
|---|---|---|---|---|---|
| | (dose I + II) | | (CMC-only) | | |
| Characteristic | N = 68 | | N = 34 | | P-value |
| Weight end treatment (g) | | | | | 0.027 |
| Mean | 28.7 | | 29.9 | | |
| SD | 2.37 | | 3.07 | | |
| Adenomyosis (n, %) | | | | | 0.768 |
| No | 10 | (14.9%) | 4 | (11.8%) | |
| Yes | 57 | (85.1%) | 30 | (88.2%) | |
| Missing | | | | | |
| Grade (n, %) | | | | | 0.048* |
| 0/1 | 27 | (40.3%) | 7 | (20.6%) | |
| 2/3 | 40 | (59.7%) | 27 | (79.4%) | |
| Missing | 1 | | 0 | | |
| Ectopic areas | | | | | 0.607 |
| Median, [range] | 1.3 | [0-5] | 1.6 | [0-7] | |
| Missing | 4 | | 0 | | |
| Ectopic glands | | | | | 0.642 |
| Median | 2.8 | | 3.3 | | |
| Range | [0-11.8] | | [0-27.5] | | |
| Missing | 6 | | 0 | | |
| Spread | | | | | 0.858 |
| None | 10 | (15.9%) | 4 | (11.8%) | |
| Focal | 19 | (30.2%) | 11 | (32.4%) | |
| Diffuse | 34 | (54.0%) | 19 | (55.9%) | |
| Missing | 5 | | 0 | | |
| Uteri parameters (α-SMA) median, IQR [p25-p75] | | | | | |
| Ratio thinnest myometrium/ | 0.24, 17 | [0.19-0.36] | 0.19, 0.15 | [0.13-0.28] | 0.023* |
| thickest myometrium | | | | | |
| Ratio thinnest myometrium/thinnest uterine wall | 0.20, 0.13 | [0.14-0.27] | 0.16, 0.10 | [0.13-0.23] | 0.174 |
| Ratio thickest myometrium/thickest uterine wall | 0.52, 0.17 | [0.43-0.59] | 0.55, 0.11 | [0.51-0.63] | 0.015* |
| Ratio thickest myometrium/transverse | 0.18, 0.06 | [0.15-0.21] | 0.20, 0.07 | [0.17-0.24] | 0.007* |

uterine diameter

Abbreviations: SD, standard deviation; En, endometrium; M, myometrium.
* significant p < 0.05

The staining of Vimentin aided in the grading of the adenomyosis per uterine section (see example FIG. 4). While the HE staining was the primary tool to assess the presence of ectopic endometrium glands, α-SMA staining demarcated the location of myometrium interruption and Vimentin staining depicted whether this interruption was endometrium stroma (FIG. 4).

### Discussion

Angiogenesis inhibition in tamoxifen-induced adenomyosis in a murine model through the administration of anti-VEGFR2 tyrosine kinase inhibitor leads to a reduction in the severity of adenomyosis. Adenomyosis grade 0 and 1 were more prevalent in the groups treated with axitinib dose I/II, while grade 2 and 3 are more prevalent in the placebo group (p=0,048; odds ratio 1.33). Furthermore, the residual unaffected myometrium was thinner at the affected site and the myometrium adjacent to the affected site was thicker in the placebo-treated group. Together with a lower ratio of thinnest to thickest myometrium, these results indicate more severe myometrium infiltration and hypertrophy in the placebo—than in the axitinib-treated groups. These results support our hypothesis that angiogenesis inhibition is a promising therapeutic approach in adenomyosis.

Other studies using the tamoxifen-induced adenomyosis murine model have been reported. A study by Jin et al (Jin, Wu et al. 2020) investigated the therapeutic effect of NSAIDs in the same model as was used in the present study and found that both NSAIDs and celecoxib reduced the depth of endometrial infiltration in the myometrium. Celecoxib, which is a selective COX-2 inhibitor, demonstrated the greatest effect and in addition had an analgesic effect by prolonging the thermal response latency. However, only the naproxen treated mice demonstrated a significant reduction in vessel density. While the effect of inhibition of COX-2 on angiogenesis has also been investigated in the treatment of cancer, the association between COX-2 inhibition and angiogenesis has not been fully established. The authors of this study suggest that their observed reduction in adenomyosis is due to celecoxib's effect on the suppression of estrogen production demonstrated by the lower concentration of estrogen and aromatase P450, reversing epithelial-to-mesenchymal transition and reducing uterine fibrosis. Important to note is that Jin et al started the NSAID treatment at the age of day 6, while it has been described that neonatal tamoxifen exposure leads to adenomyosis by 6 weeks of age and at 10 days at the earliest (Mehasseb, Bell et al. 2009). Starting treatment before that time might prevent rather than treat adenomyosis in mice.

Zhu et al (Zhu, Chen et al. 2016) also used the tamoxifen-induced mice model, but used it to test Ozagrel treatment, which is an anti-mouse GPIbα polyclonal IgG antibody that depletes platelets, from week 16 of age. They found that platelet depletion lead to suppressed myometrial infiltration, improved hyperalgesia, reduced uterine contractility, lowered plasma corticosterone levels and slower down fibrogenesis. Although these results are promising and point towards the involvement of increased platelet aggregation in adenomyosis, the clinical application of anti-platelet therapy is limited due to the hemorrhage risk.

Other models have been used in studies aimed at investigating adenomyosis treatment. Zhou et al (Zhou, Mori et al. 2003) implanted a single pituitary gland in SHN mice at 7 weeks of age. Treatment started one day after pituitary grafting until week 13 using TNP-470, a fumagillin analogue that inhibits endothelial cell migration and proliferation. The TNP-470-treated group did not develop signs of uterine adenomyosis as opposed to 80% in the control group, and their mean surface area of blood vessels in the endometrium reduced to 60.5% of that in the control group. Although Zhou et al. reports that the mice are reported to develop adenomyosis spontaneously, the publication has an unclear definition of adenomyosis and the results cannot be interpreted as there is no description of the assessment used. Huang et al (Huang, Chen et al. 2014) found that treatment with bevacizumab (anti-VEGF monoclonal antibody), administered 14 days after ovariectomized mice were xenografted with human adenomyosis lesions at week eight, decreased the MVD and the expression of VEGF. In a similar model, Zhou et al (Zhou, Yi et al. 2012), demonstrated that knockdown of annexin A2 (ANXA2), an estrogen-responsive protein, reduced angiogenesis, suggesting its potential as therapeutic target. However, both studies using the xenotransplantation method are incomparable to the present study, since the adenomyosis was xenotransplanted intraperitoneally, and thereby becomes endometriosis. Neither study evaluated the effect on adenomyosis in utero. The lesions are thus present outside of the uterus and the mice do not develop adenomyosis, but rather a form of endometriosis.

In contrast, the present study is performed using a mouse model of adenomyosis. As will be recognized by a skilled person, angiogenic processes are quite different in the uterus as compared to other tissues. Angiogenesis in adults is rare except in certain pathological conditions such as wound healing and solid tumor growth. One exception is the angiogenesis that occurs in the uterus. In contrast to uncontrolled vascular growth associated with tumor growth, angiogenesis in the uterus is controlled and cyclical.

The strength of the present study is the optimal study design, in which the most accurate adenomyosis animal model was chosen and validated before starting the experiment. Also, an appropriate sample size was used in order to reach relevant and reliable study outcomes. Since treatment was started only after adenomyosis was certainly induced, this study represents outcomes that are eventually applicable in a clinical setting, where women will seek treatment for adenomyosis only after the diagnosis has been established. This way, a full eradication of the adenomyosis would probably not have been feasible, but since this study was powered to evaluate a clinical relevant reduction of adenomyosis instead, the present results are promising and reliable.

### Conclusion & Future Perspectives

In conclusion, the inhibition of angiogenesis in adenomyosis reduced the growth of ectopic endometrium, which might eventually alleviate symptoms of abnormal uterine bleeding and subfertility when applied in a clinical setting. Since there is currently no treatment option for premenopausal women who wish to retain their uterus and their fertility, treatment with an angiogenesis inhibitor might provide a solution. The major requirement in further development of anti-angiogenesis therapy is a safety and side-effect profile tolerable for this population. Since angiogenesis has an important function in ovarian function and regeneration in the menstrual cycle, the application would need to be selective so that physiological functions are preserved. On the other hand, anti-angiogenesis therapy might even be effective by applying it for a certain time period until ectopic endometrium has diminished. Since the FDA advices to wait one month after discontinuing axitinib therapy until trying to conceive, a short non-therapeutic period might allow safe conception and gestation. To further optimize the treatment potential of an angiogenesis inhibitor for adenomyosis, local administration in utero would be most promising. By limiting the systemic exposure, a lower dose might inhibit angiogenesis at the affected site without toxic side-effects on the ovaries for example.

APPENDIX TABLE I
Characteristics of mice in validation experiment

| | Vehicle | | Tamoxifen | | |
|---|---|---|---|---|---|
| Characteristic | (n = 6) | | (n = 6) | | P-value |
| Weight | | | | | |
| Median | 25.5 | | 25.2 | | 0.936 |
| Range | 22.7-28.8 | | 23.1-29.8 | | |
| Adenomyosis | | | | | |
| No | 6 | (100%) | 0 | (0%) | |
| Yes | 0 | (0%) | 6 | (100%) | |
| Grade | | | | | |
| 0 | 6 | (100%) | 0 | (0%) | |
| 1 | 0 | (0%) | 0 | (0%) | |
| 2 | 0 | (0%) | 6 | (100%) | |
| 3 | 0 | (0%) | 0 | (0%) | |
| Ectopic glands | | | | | |
| Median | 0 | | 7.5 | | |
| Range | 0 | | 3-15 | | |
| Spread | | | | | |
| Focal | 0 | (0%) | 2 | (33.3%) | |
| Diffuse | 0 | (0%) | 4 | (66.7%) | |
| Microvessel density, median [range] | | | | | |
| Endometrium | 5.3 | [4.1-7.0] | 5.9 | [5.0-6.8] | 0.420 |
| Ectopic endometrium | — | | 4.8 | [4.0-6.0] | — |
| Myometrium | 3.0 | [2.4-3.6] | 3.0 | [2.5-4.0] | 0.699 |

| | | | |
|---|---|---|---|
| Uteri parameters (α-SMA) | | | |
| Ratio thinnest myometrium/thinnest uterine wall | 0.58 | 0.18 | 0.004 |
| Ratio thickest myometrium/thickest uterine wall | 0.40 | 0.43 | 1.000 |
| Ratio thinnest myometrium/thickest | 0.65 | 0.19 | 0.004 |
| myometrium | | | |

### Example 1.1 Reduced Adenomyosis Severity Index after Treatment with Axitinib in Low Dose

### Methods

The adenomyosis severity index in tamoxifen-induced adenomyosis in a murine model has been determined based on the abovementioned results of grade of adenomyosis and adenomyosis-affected surface area. Specifically, the grade of adenomyosis (determined on H&E slides as described above) was multiplied by the adenomyosis-affected surface area (determined on vimentin-stained slides). The adenomyosis severity index is the average of all sections analyzed per uterine horn. The adenomyosis severity index was determined for placebo group (CMC-treated mice) and two axitinib-treated groups (dose II—AX3: axitinib 3 mg/kg; dose I—AX25: axitinib 25 mg/kg).

### Results

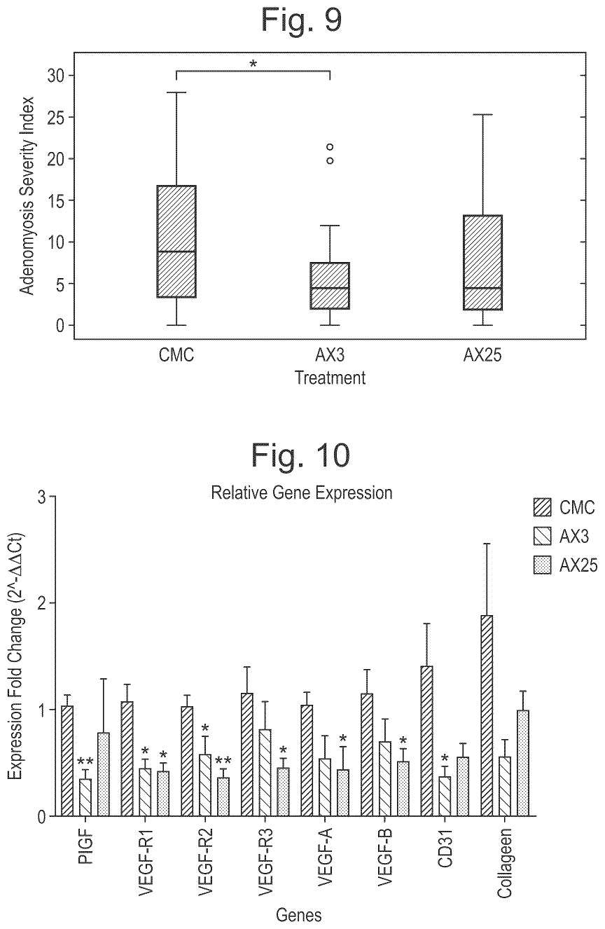

The adenomyosis severity index was reduced by 48% in the mice treated with axitinib dose II (3 mg/kg) compared to the placebo-treated mice (difference between groups Kruskal-Wallis=0.07, post hoc difference between placebo- and AX3-treated (dose II) group p<0.05 and between placebo and AX25-treated (dose I) group p=0.173) (FIG. 9). These result thus show that low dose of axitinib successfully reduced adenomyosis severity index of tamoxifen-exposed mice.

### Example 1.2 Uterine Gene Expression Profile after Treatment with Axitinib

### Methods

To study the efficacy of axitinib treatment at the molecular level, the expression levels of vascular marker CD31 and angiogenic growth factors VEGF-A, VEGF-B, VEGF receptor R1 (VEGF-R1), VEGF-R2, VEGF-R3, placental growth factor PlGF, and collagen were measured. Real-time quantitative PCR was performed in a random selection of uterine specimens exposed to axitinib (dose II, n=7; dose I, n=6) or placebo (CMC, n=7). The expression of each gene was normalized to the household gene cyclophilin A.

### Results

Treatment with dose II axitinib resulted in a down-regulation of VEGF-R1 by 63% (p=0.003), and of VEGF-R2 by 45% (p=0.038). Treatment with dose I axitinib resulted in down-regulation of all VEGF receptors (1-3), as well as VEGF-A and VEGF by more than 60% (p<0.05). This indicates that Axinitib, as a VEGF blocker, indeed modulates angiogenesis signalling pathway, which can thus result in reduced angiogenesis. Also the expression of the endothelial cell marker CD31 decreased by 85% after treatment with dose II or I axitinib (p=0.04 and p=0.06, FIG. 10), confirming the reduced level of angiogenesis.

### Example 2

While increased angiogenesis appears to be the underlying mechanism allowing the disease to develop in early stages, preliminary results indicate that fibrosis is increased in late-stage adenomyosis (Trommelen et al. 2022). Therefore, earlier application of antiangiogenic treatment is expected to increase the effect of this therapy.

The following example uses mouse model for adenomyosis as described previously. In short, neonatal mice will be orally dosed with tamoxifen (1 mg/kg) at day 2-5 after birth and adenomyosis is allowed to develop until the age of six weeks.

### Determining Optimal Timing to Start Anti-Angiogenesis Therapy

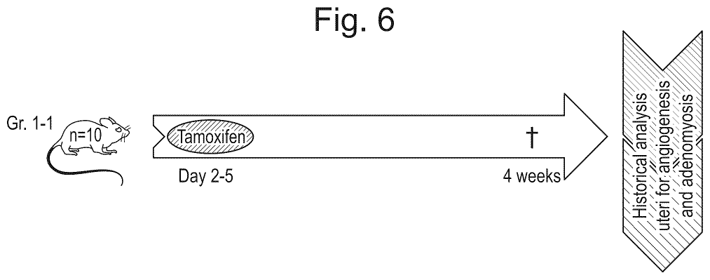

In our previous mice experiments we allowed the tamoxifen-induced adenomyosis to develop for six weeks. However, using histological examination, it is noticed that the adenomyosis is in an advanced stage characterized by signs of fibrosis. Anti-angiogenesis therapy would be more effective in an earlier stage characterized by increased angiogenesis only. Therefore, uteri of tamoxifen-treated mice are examined after 4 weeks (FIG. 6). The level of angiogenesis (CD31 staining), adenomyosis (HE staining), and fibrosis (PTAH) of uteri obtained 4 weeks post-tamoxifen treatment are analysed, and compared with uteri obtained 6 weeks post-tamoxifen treatment.

### Intra-Uterine Administration of Axitinib

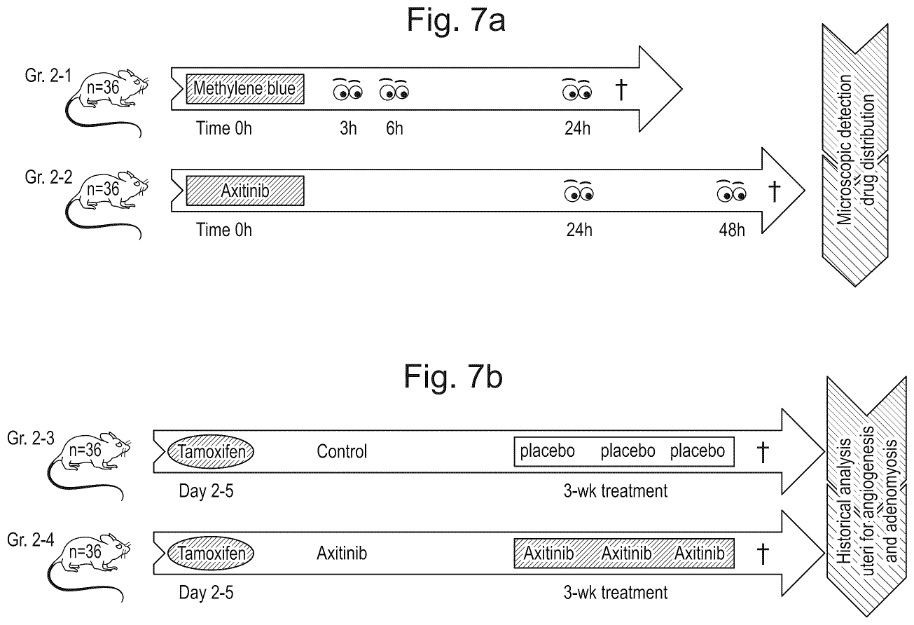

Intra-uterine administration is performed using a specialized catheter and axinitib is deposited in one uterine horn, while the other horn serves as an internal control. The experiments are divided into two types; short-term and long-term experiments (FIG. 7).

First, the short-term experiments are performed to demonstrate proof-of-principle of intra-uterine delivery. Visual detection of the applied solvent after 3, 6 and 24 hours in live mice is studied. When axitinib is replaced by a model drug, such as methylene blue, uptake and distribution in uterine tissue can be microscopically assessed after termination. Different dosages of axitinib can be applied, the presence of axinitib solvent is be assessed after 48 hours, as well as the direct effect of axitinib on the endo- and myometrium in the treated horn compared to the non-treated horn. Long-term experiments encompass intra-uterine administration of axinitib for a period of three weeks. After terminating the mice, the level of angiogenesis is assessed as well as the level of adenomyosis and fibrosis.

### Effect of Adenomyosis and Axitinib Therapy on Fertility

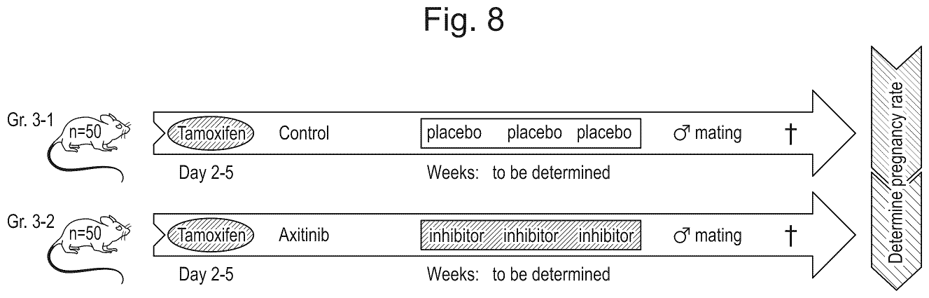

The putative effect that adenomyosis may have on fertility is studied in mice by determining pregnancy rates. Tamoxifen-treated mice are mated after axitinib treatment (FIG. 8). Pregnancy rates are determined after one mating attempt and compared with pregnancy rates for control mice. The potential effect of axinitib treatment on fertility is also examined.

We expect that anti-angiogenesis therapy is able to stop the progression of the disease in an early stage and offer an essential solution for women in their child baring age. We hypothesize that local treatment will further enhance the therapeutic potential of angiogenesis inhibitors, while a lower dosage can be administered.

### Example 3—Intra-Uterine Administration of Sunitinib

Sunitinib is another angiogenesis inhibitor and a highly-fluorescent molecule, therefore it will be used as a model drug to microscopically assess uptake and distribution in uterine tissue after termination. It is expected that a 100-1000× lower dosage (1% and 0.1% of systemic dose) can be successfully injected in the uterus of mice, with uptake throughout the myometrium observed 4 h and 24 h after injection.

Intra-uterine delivery of a gel containing slow-release Sunitinib in low dose will also be tested as an alternative treatment for oral Axinitib in mice with tamoxifen-induced adenomyosis. It is expected that a low dose (1% and/or 0.1%) of Sunitinib is already potent in inhibiting angiogenesis when delivered locally instead of systemically, without any toxic effects.

### REFERENCES

Bird, C. C., T. W. McElin and P. Manalo-Estrella (1972). “The elusive adenomyosis of the uterus—revisited.” Am J Obstet Gynecol 112(5): 583-593.
Castermans, K., S. P. Tabruyn, R. Zeng, J. R. van Beijnum, C. Eppolito, W. J. Leonard, P. A. Shrikant and A. W. Griffioen (2008). “Angiostatic activity of the antitumor cytokine interleukin-21.” Blood 112(13): 4940-4947.
Critchley, H. O. D., E. Babayev, S. E. Bulun, S. Clark, I. Garcia-Grau, P. K. Gregersen, A. Kilcoyne, J. J. Kim, M. Lavender, E. E. Marsh, K. A. Matteson, J. A. Maybin, C. N. Metz, I. Moreno, K. Silk, M. Sommer, C. Simon, R. Tariyal, H. S. Taylor, G. P. Wagner and L. G. Griffith (2020). “Menstruation: science and society.” Am J Obstet Gynecol 223(5): 624-664.
Critchley, H. O. D., J. A. Maybin, G. M. Armstrong and A. R. W. Williams (2020). “Physiology of the Endometrium and Regulation of Menstruation.” Physiol Rev 100(3): 1149-1179.
Dings, R. P., X. Chen, D. M. Hellebrekers, L. I. van Eijk, Y. Zhang, T. R. Hoye, A. W. Griffioen and K. H. Mayo (2006). “Design of nonpeptidic topomimetics of antiangiogenic proteins with antitumor activities.” J Natl Cancer Inst 98(13): 932-936.
Folkman, J. (1995). “Angiogenesis in cancer, vascular, rheumatoid and other disease.” Nat Med 1(1): 27-31.
Green, A. R., J. A. Styles, E. L. Parrott, D. Gray, R. E. Edwards, A. G. Smith, T. W. Gant, P. Greaves, F. Al-Azzawi and I. N. White (2005). “Neonatal tamoxifen treatment of mice leads to adenomyosis but not uterine cancer.” Exp Toxicol Pathol 56(4-5): 255-263.
Griffioen, A. W. and G. Molema (2000). “Angiogenesis: potentials for pharmacologic intervention in the treatment of cancer, cardiovascular diseases, and chronic inflammation.” Pharmacol Rev 52(2): 237-268.
Harmsen, M. J., A. Arduc, L. J. M. Juffermans, A. W. Griffioen, E. S. Jordanova and J. A. F. Huirne (2022). “Evidence of increased angiogenesis, and lympangiogenesis in adenomyosis.” Submitted.
Harmsen, M. J., C. F. C. Wong, V. Mijatovic, A. W. Griffioen, F. Groenman, W. J. K. Hehenkamp and J. A. F. Huirne (2019). “Role of angiogenesis in adenomyosis-associated abnormal uterine bleeding and subfertility: a systematic review.” Hum Reprod Update 25(5): 647-671.
Hinz, B. (2007). “Formation and function of the myofibroblast during tissue repair.” J Invest Dermatol 127(3): 526-537.
Huang, T. S., Y. J. Chen, T. Y. Chou, C. Y. Chen, H. Y. Li, B. S. Huang, H. W. Tsai, H. Y. Lan, C. H. Chang, N. F. Twu, M. S. Yen, P. H. Wang, K. C. Chao, C. C. Lee and M. H. Yang (2014). “Oestrogen-induced angiogenesis promotes adenomyosis by activating the Slug-VEGF axis in endometrial epithelial cells.” J Cell Mol Med 18(7): 1358-1371.
Jin, Z., X. Wu, H. Liu and C. Xu (2020). “Celecoxib, a selective COX-2 inhibitor, markedly reduced the severity of tamoxifen-induced adenomyosis in a murine model.” Exp Ther Med 19(5): 3289-3299.
Kang, S., S. Z. Li, N. Wang, R. M. Zhou, T. Wang, D. J. Wang, X. F. Li, J. Bui and Y. Li (2010). “Association between genetic polymorphisms in fibroblast growth factor (FGF)1 and FGF2 and risk of endometriosis and adenomyosis in Chinese women.” Hum Reprod 25(7): 1806-1811.
Leyendecker, G., L. Wildt and G. Mall (2009). “The pathophysiology of endometriosis and adenomyosis: tissue injury and repair.” Arch Gynecol Obstet 280(4): 529-538.
Li, B., M. Chen, X. Liu and S. W. Guo (2013). “Constitutive and tumor necrosis factor-α-induced activation of nuclear factor-KB in adenomyosis and its inhibition by andrographolide.” Fertil Steril 100(2): 568-577.
Liu, X., M. Shen, Q. Qi, H. Zhang and S. W. Guo (2016). “Corroborating evidence for platelet-induced epithelial-mesenchymal transition and fibroblast-to-myofibroblast transdifferentiation in the development of adenomyosis.” Hum Reprod 31(4): 734-749.
Martinez-Conejero, J. A., M. Morgan, M. Montesinos, S. Fortuno, M. Meseguer, C. Simon, J. A. Horcajadas and A. Pellicer (2011). “Adenomyosis does not affect implantation, but is associated with miscarriage in patients undergoing oocyte donation.” Fertil Steril 96(4): 943-950.
Mehasseb, M. K., S. C. Bell and M. A. Habiba (2009). “The effects of tamoxifen and estradiol on myometrial differentiation and organization during early uterine development in the CD1 mouse.” Reproduction 138(2): 341-350.
Mori, T. and H. Nagasawa (1983). “Mechanisms of development of prolactin-induced adenomyosis in mice.” Acta Anat (Basel) 116(1): 46-54.
Pontis, A., M. N. D′Alterio, S. Pirarba, C. de Angelis, R. Tinelli and S. Angioni (2016). “Adenomyosis: a systematic review of medical treatment.” Gynecol Endocrinol 32(9): 696-700.
Rosenfeld, P. J., D. M. Brown, J. S. Heier, D. S. Boyer, P. K. Kaiser, C. Y. Chung, R. Y. Kim and M. S. Group (2006). “Ranibizumab for neovascular age-related macular degeneration.” N Engl J Med 355(14): 1419-1431.
Sugino N, Kashida S, Karube-Harada A, Takiguchi S, Kato H. Expression of vascular endothelial growth factor (VEGF) and its receptors in human endometrium throughout the menstrual cycle and in early pregnancy. Reproduction 2002; 123:379-387.
Tadayoni, R., L. Sararols, G. Weissgerber, R. Verma, A. Clemens and F. G. Holz (2021). “Brolucizumab: A Newly Developed Anti-VEGF Molecule for the Treatment of Neovascular Age-Related Macular Degeneration.” Ophthalmologica 244(2): 93-101.
Taran, F. A., E. A. Stewart and S. Brucker (2013). “Adenomyosis: Epidemiology, Risk Factors, Clinical Phenotype and Surgical and Interventional Alternatives to Hysterectomy.” Geburtshilfe und Frauenheilkunde 73(9): 924-931.
Thairu, N., S. Kiriakidis, P. Dawson and E. Paleolog (2011). “Angiogenesis as a therapeutic target in arthritis in 2011: learning the lessons of the colorectal cancer experience.” Angiogenesis 14(3): 223-234.
Thiery, J. P., H. Acloque, R. Y. Huang and M. A. Nieto (2009). “Epithelial-mesenchymal transitions in development and disease.” Cell 139(5): 871-890.
   Thijssen, V. L., R. Postel, R. J. Brandwijk, R. P. Dings, I. Nesmelova, S. Satijn, N. Verhofstad, Y. Nakabeppu, L. G. Baum, J. Bakkers, K. H. Mayo, F. Poirier and A. W. Griffioen (2006). “Galectin-1 is essential in tumor angiogenesis and is a target for antiangiogenesis therapy.” Proc Natl Acad Sci USA 103(43): 15975-15980.
Tremellen, K. and P. Russell (2011). “Adenomyosis is a potential cause of recurrent implantation failure during IVF treatment.” Aust N Z J Obstet Gynaecol 51(3): 280-283.
Trommelen, L., M. J. Harmsen, R. A. de Leeuw, T. van den Bosch and J. A. F. Huirne (2022). “Adenomyosis: a spectrum of stadia? Hypotheses on the etiology and manifestations of adenomyosis.” manuscript-in-preparation.
Vannuccini, S., C. Tosti, F. Carmona, S. J. Huang, C. Chapron, S. W. Guo and F. Petraglia (2017). “Pathogenesis of adenomyosis: an update on molecular mechanisms.” Reprod Biomed Online 35(5): 592-601.
Vercellini, P., D. Consonni, D. Dridi, B. Bracco, M. P. Frattaruolo and E. Somigliana (2014). “Uterine adenomyosis and in vitro fertilization outcome: a systematic review and meta-analysis.” Hum Reprod 29(5): 964-977.
Wang, J., X. Deng, Y. Yang, X. Yang, B. Kong and L. Chao (2016). “Expression of GRIM-19 in adenomyosis and its possible role in pathogenesis.” Fertil Steril 105(4): 1093-1101.
Wentink, M. Q., T. M. Hackeng, S. P. Tabruyn, W. C. Puijk, K. Schwamborn, D. Altschuh, R. H. Meloen, T. Schuurman, A. W. Griffioen and P. Timmerman (2016). “Targeted vaccination against the bevacizumab binding site on VEGF using 3D-structured peptides elicits efficient antitumor activity.” Proc Natl Acad Sci USA 113(44): 12532-12537.
Yen, C. F., S. J. Huang, C. L. Lee, H. S. Wang and S. K. Liao (2017). “Molecular Characteristics of the Endometrium in Uterine Adenomyosis and Its Biochemical Microenvironment.” Reprod Sci 24(10): 1346-1361.
Zhou, S., T. Yi, R. Liu, C. Bian, X. Qi, X. He, K. Wang, J. Li, X. Zhao, C. Huang and Y. Wei (2012). “Proteomics identification of annexin A2 as a key mediator in the metastasis and proangiogenesis of endometrial cells in human adenomyosis.” Molecular and Cellular Proteomics 11(7).
Zhou, Y. F., T. Mori, H. Kudo, R. Asakai, S. Sassa and S. Sakamoto (2003). “Effects of angiogenesis inhibitor TNP-470 on the development of uterine adenomyosis in mice.” Fertil Steril 80 Suppl 2: 788-794.
Zhu, B., Y. Chen, X. Shen, X. Liu and S. W. Guo (2016). “Anti-platelet therapy holds promises in treating adenomyosis: experimental evidence.” Reprod Biol Endocrinol 14(1): 66.

1. A non-steroidal angiogenesis inhibitor for use in treating or preventing a benign uterine disorder in a female mammalian subject, comprising locally administering to a subject in need thereof said non-steroidal angiogenesis inhibitor.

2. The inhibitor for use of claim 1, wherein the female mammalian subject is afflicted with benign uterine disorder and the method reduces symptoms, increases fertility, and/or increases clinical pregnancy rate and live birth rate.

3. The inhibitor for use of claim 1 or 2, wherein the benign uterine disorder is adenomyosis.

4. The inhibitor for use of any of the preceding claims, wherein the non-steroidal angiogenesis inhibitor is administered intra-uterine.

5. The inhibitor for use of any one of the preceding claims, wherein the non-steroidal angiogenesis inhibitor is provided by an intra-uterine device (IUD).

6. The inhibitor for use of any one of the preceding claims, wherein the non-steroidal angiogenesis inhibitor is provided as an extended-release formulation or via a extended-release IUD.

7. The inhibitor for use of any one of the preceding claims, wherein the female mammalian subject is a human.

8. An intra-uterine device comprising a non-steroidal angiogenesis inhibitor.

9. The intra-uterine device of claim 8, wherein the intra-uterine device further comprises progesterone or progestin.

10. The inhibitor for use or the intra-uterine device of any of the preceding claims, where in the angiogenesis inhibitor is a small molecule or an antigen binding molecule such as an antibody or antigen binding fragment thereof.

11. The inhibitor for use or the intra-uterine device of any of the preceding claims, wherein the angiogenesis inhibitor targets an angiogenic signaling axis, e.g. VEGF, VEGF receptor, EGF, EGF receptor, PDGF, PDGF receptor, or PGF, PGF receptor.

12. The inhibitor for use or the intra-uterine device of any of the preceding claims, wherein the angiogenesis inhibitor targets an angiogenesis-associated molecule or receptor, e.g. an integrin, CD36, CD44, extracellular vimentin, fibrillin-2, secreted frizzled-related protein-2, lysyl oxidase, prostate specific membrane antigen, versican, apelin.

13. The inhibitor for use or the intra-uterine device of any of the preceding claims, wherein the angiogenesis inhibitor is an anti-VEGF binding molecule such as an antibody or antigen binding fragment thereof, a peptibody or a nanobody.

14. The inhibitor for use or the intra-uterine device of claim 13, wherein the angiogenesis inhibitor is selected from bevacizumab, ramucirumab, or ranibizumab.

15. The inhibitor for use or the intra-uterine device of claim 10, wherein the small molecule is a tyrosine kinase inhibitor, preferably selected from axitinib, imatinib, erlotinib, cabozantinib, lapatinib, pazopanib, ponatinib, regorafenib, sunitinib, sorafenib, vandetanib, and crizotinib and/or a pharmaceutically acceptable salt thereof.
